(19) United States
(12) Patent Application Publication
(10) Pub. No.: US 2005/0170445 A1
(43) Pub. Date: Aug. 4, 2005

(54) Methods of establishing profiles for use in evaluating wound healing and biocompatibility of implant materials and microarrays useful therefor

(75) Inventors: William Monty Reichert, Hillsborough, NC (US); Yiwen Li, Durham, NC (US)

Correspondence Address: JENKINS, WILSON & TAYLOR, P. A., 3100 TOWER BLVD, SUITE 1400, DURHAM, NC, 27707, US

(73) Assignee: Duke University

(21) Appl. No.: 11/031,552

(22) Filed: Jan. 7, 2005

Related U.S. Application Data
(60) Provisional application No. 60/534,814, filed on Jan. 7, 2004.

(51) Int. Cl. G01N 33/53; G01N 33/537; G01N 33/543

(52) U.S. Cl. 435/7.92

(57) ABSTRACT

Protein detection microarrays are used to specifically detect cytokines and growth factors that are associated with wound healing and with host organism responses to foreign, implanted materials (i.e., biocompatibility). Methods of establishing profiles that can be used in evaluating wound healing and biocompatibility take advantage of such cytokine- and growth factor-specific microarrays, which comprise anti-cytokine and anti-growth factor capture antibodies immobilized onto a solid substrate. The microarrays are produced using a printing buffer optimized for cytokine/growth factor detection. Methods of detecting the cytokines and growth factors also utilize optimized blocking buffers, fluorescent dyes, and immunoassay conditions. Sandwich and direct label fluoroimmunoassays can be carried out with the optimized microarrays. Kits for establishing profiles that can be used in the evaluation of wound healing and biocompatibility comprise cytokine- and growth factor-specific microarrays, and optionally include buffers suitable for detection immunoassays, including optimized printing buffers and blocking buffers.

### CROSS-REFERENCE TO RELATED APPLICATION

This application claims the benefit of U.S. Provisional Patent Application Ser. No. 60/534,814, filed Jan. 7, 2004, the disclosure of which is incorporated herein by reference in its entirety.

### GRANT STATEMENT

This presently disclosed subject matter was supported by grant HL/DK 54932 from the National Institutes of Health. Thus, the United States government has certain rights in the presently disclosed subject matter.

### TECHNICAL FIELD

The presently disclosed subject matter relates generally to the use of high-throughput protein microarrays for the detection of cytokines and growth factors associated with wound healing and host-organism response to bioimplantation.

### Table of Abbreviations

APO-1—apoptosis antigen-1

ATCC—American Type Culture Collection

BCA—bicinchoninic acid

BSA—bovine serum albumin

cDNA—complementary DNA

DMSO—dimethyl sulfoxide

deg—degrees

dUTP—2′-deoxyuridine 5′-triphosphate

ECL—electrochemiluminescence

EDTA—ethylenediaminetetraacetic acid

EGF—epidermal growth factor

ELISA—enzyme-linked immunosorbent assay

Fab—antigen-binding antibody fragment

FBS—fetal bovine serum

FCS—Fluorescence Correlation Spectroscopy

FGF—fibroblast growth factor

Fv—antigen-binding antibody fragment

FDA—Food and Drug Administration

FITC—fluorescein isothiocyanate

G-CSF—granulocyte colony simulating factor

GM-CSF—granulocyte macrophage growth factor

GRO—growth-related protein

GSF—glia cell stimulating factor

h or hr—hours

I-309—human CC cytokine

IFN—interferon

Ig—immunoglobulin

IgG—immunoglobulin G

IL-1—interleukin 1

IL-1ra—interleukin-1 receptor antagonist

IL-12—interleukin 12

IL-13—interleukin 13

IL-2—interleukin 2

IL-4—interleukin 4

IL-7—interleukin 7

L—liters

LPS—lipopolysaccharide

mAb—monoclonal antibody

MCP—monocyte chemotactic protein

MDC—macrophage-derived chemokine

MIG—monokine induced by IFN-γ

MIP—macrophage inflammatory protein

mg—milligrams

mL—milliliters

mmol—millimoles

μg—micrograms

μL—microliters

μM—micromolar

ng—nanograms

nL—nanoliters

nM—nanomolar

PBS—phosphate buffered saline

PDGF—platelet derived growth factor

PCR—polymerase chain reaction

PE—phycoerythrin

PES—polyethersulfone

PET—positron emission tomography

pg—picograms

pL—picoliters

pmol—picomoles

RANTES—regulated upon activation, normal T-cell expressed and secreted

RT-PCR—reverse transcription-polymerase chain

SA—streptavidin

SAA—serum amyloid A

sICAM—soluble intercellular adhesion molecule

sVCAM—soluble vascular cell adhesion molecule

TBS—Tris-buffered saline

TGF—transforming growth factor

THP-1—a human monocyte leukemia cell line; ATCC No. TIB-202

TNF—tissue necrosis factor

VEGF—vascular endothelial growth factor

### BACKGROUND

Macrophages play an important role in the inflammation process, and are also thought to mediate a host of reparative cellular events, such as endothelial cell, fibroblast, and smooth muscle cell proliferation, stimulation of collagen synthesis, and activation and recruitment of lymphocytes, leukocytes, and platelets. J. M. Anderson and K. M. Miller, Biomaterials (1984) 5, 5-10; A. J. Singer and R. A. F. Clark, New England Journal of Medicine (1999) 341, 738-746; R. Gillitzer and M. Goebeler, Journal of Leukocyte Biology (2001) 69, 513-521; E. Lin et al., Surgery (2000) 127, 117-126. The molecules released by macrophages that orchestrate reparative events are an array of interleukins, interferons, and growth factors that bind to membrane receptors of neighboring cells and either enhance or inhibit function. Immune mediators produced in response to a stimulus are generically referred to as cytokines. Growth factors tend to be produced constitutively; however, some mediators originally designated as growth factors also act as cytokines.

Wound healing is a complicated molecular process that normally progresses through a series of distinct stages. These healing stages include, progressively, inflammation, cellular proliferation, repair, and maturation. Inflammation is the first biochemical response of the body to a wound, and is known to be mediated by complex pathways of signaling proteins. During proliferation, monocytes and macrophages produce growth factors that attract fibroblasts and endothelial cells to the wound, stimulate the production of collagen, and establish blood supply. During the third phase, repair, the wound is covered by scar tissue as the surface area of the wound continues to decrease. In the final stage, maturation, the scar tissue is remodeled and becomes comparable to normal tissue.

Early and accurate evaluation of inflammation and the later stages of wound healing allow the medical practitioner to begin appropriate therapeutic regimens. Improperly diagnosed wounds such as diabetic ulcers, venous-stasis wounds, and pressure sores frequently result in negative patient outcomes. Unfortunately, the diagnosis and evaluation of wounds is generally empirical, largely visual, and often imprecise or insufficiently accurate. Visual wound assessments, in particular, have proven to be inadequate for proper diagnosis, especially for chronic wounds. There is thus a need for improved methods and techniques for the diagnosis and evaluation of wound healing as it progresses over time.

Cytokines and growth factors are essential promoters and mediators of proper wound healing. See J. M. Anderson and K. M. Miller (1984), and E. Lin et al., (2000), 127, 117-126. Specific cytokines and/or growth factors are thought to be associated with particular time points in wound healing, and there is significant variation in the amount of cytokine and/or growth factor present at various stages of the healing process. Accordingly, the ability to generate and monitor a temporal profile of cytokine/growth factor levels present in a wound would provide the medical practitioner with a valuable wound healing diagnostic asset. However, wound cytokine/growth factor levels are transient and occur at very low concentrations (e.g., on the order of picograms per milliliter). Reliable and accurate approaches for detecting the concentrations of cytokines/growth factors in healing wounds are not provided by currently used protein detection protocols, and have heretofore not been disclosed.

Cytokines and growth factors also play significant roles in the mediation of host organism responses to materials and devices that are implanted or inserted into living host organisms (e.g., bioimplantation materials and devices such as glucose sensors). When a biomaterial article is implanted into an organism, host-defense mechanisms are initiated. This host response is controlled and modulated by macrophage-derived cytokines and growth factors. The types and levels of cytokines and growth factors surrounding a biomaterial can initially drive the acute and chronic inflammatory reactions, and can later induce the wound healing response while inflammation resolves. Macrophages are the major inflammatory cell type found on the surface of biomaterials, and, together with monocytes, are known to play a critical role in the biological response to implanted materials. Because monocytes direct much of the chronic inflammatory response, the ability of a biomaterial to alter cell viability or secretory function can have significant consequences to the overall biological response of the biomaterial.

In particular, it is believed that inflammatory host organism responses are closely related to the implant-related sensor failure mechanisms of membrane biofouling and tissue encapsulation. For longer term percutaneous or totally implantable devices, the ability to rigorously assess in vivo tissue-sensor interactions is a beneficial aspect of a greater understanding of this response mechanism.

In general, assessment of sensor biocompatibility mainly includes examining the effects of four processes on sensor performance: protein adsorption, cellular adhesion, inflammation, and composition of the encapsulating tissue. These four processes are closely linked phenomenologically. Protein adsorption and cellular adhesion are primarily associated with the clotting and fibrin formation of hemostasis. Infiltration of leukocytes into the wound healing bed and proliferation of macrophages dominates the inflammation that occurs in the first few days of wound healing. If the wound heals acutely, then inflammation gradually gives way to a vascularized granulation tissue, which after a week to ten days is replaced by an increasingly avascular capsular tissue.

Many new biomaterials or devices fail in animal tests or human clinical trials because of inadequate compatibility with host tissue. Improved in vitro pre-screening of biomaterials could significantly reduce the cost and uncertainty of developing new biomaterials. In vitro models tend to be sensitive and specific, because effects are likely to be exaggerated, due to concentration effects and the absence of protective mechanisms present in the intact animal. Techniques currently available in the art typically allow investigators to study only one or a few inflammation mediators (e.g., cytokines or growth factors) at one time. At present, no reliable approach exists for the temporal profiling of cytokine and growth factor content of tissues or cells under insult, whether such insult is caused by a wound or by a host organism response to implantation of a foreign material.

Present methods for the analysis of cytokine/growth factor activity in blood samples include quantitative RT-PCR and ELISA. Quantitative RT-PCR measures the level of cytokine mRNA in cells of interest. This method requires the extraction of mRNA from cells and thus requires a great deal of pre-assay preparation. The rate-determining step of this assay is very resource-intensive, requiring the expensive RT-PCR instrument, which severely limits scaling up the throughput of this method. Currently, only about two cytokines can be detected per assay reaction with quantitative RT-PCR, which also limits the potential throughput of this assay. ELISA methods directly measure the concentration of cytokine protein in serum or cell supernatants. ELISA assays thus require no extensive pre-assay preparation. However, these assays can only measure the concentration of one cytokine per reaction, which greatly limits their throughput. Because of the large number of cytokines and growth factors involved in complex processes such as wound healing and host organism response to implanted materials, cytokine/growth factor detection methods such as RT-PCR and ELISA are severely limited in a clinical setting.

Sensitive high throughput methods for the detection of cytokines and growth factors associated with wound healing and host organism/biocompatibility response remain highly desirable. As a general consideration, increasing throughput is generally afforded by miniaturizing and automating known assay methods. Miniaturization and automation facilitate high-throughput abilities such as the ability to process many assays simultaneously, the ability to conduct a high number of measurements simultaneously (e.g., by “scanning”), and the ability to analyze the results of such a scan quickly.

To this end, multiwell protein screening systems have been developed, with automated 96-well plate-based screening systems being the most widely used. One trend in plate-based screening systems is the ongoing reduction of reaction wells volumes, thereby increasing the density of the wells per plate (for example, going from 96-well to 384-and 1536-wells per plate). The reduction in reaction volumes results in increased throughput, dramatically decreased bioreagent costs, and a decrease in the number of plates that need to be managed by automation.

Although an increase in well numbers per plate is desirable for high throughput efficiency, the use of volumes smaller than 1 microliter in the well format generates significant problems with evaporation, dispensing times, protein inactivation, and assay adaptation. Proteins are very sensitive to the physical and chemical properties of the reaction chamber surfaces and are prone to denaturation at liquid/solid and liquid/air interfaces. Miniaturization of assays to volumes smaller than 1 microliter increases the surface to volume ratio substantially. Furthermore, solutions of submicroliter volumes evaporate rapidly, within seconds to a few minutes, when in contact with air. Maintaining microscopic volumes in open systems is therefore very difficult.

Miniaturized DNA chip technologies have been developed and are currently being exploited for nucleic acid hybridization assays. Such miniaturization, often referred to as “microarray technology”, has been evidenced by the proliferation of cDNA gene expression microarrays.

Microarray technology has, in general, not been successfully adapted to protein detection, due to differences between nucleic acid hybridization and protein-protein binding that are well characterized in the art. For example, nucleic acids withstand temperatures up to 100° C., can be dried and re-hydrated without loss of activity, and can be bound directly to organic adhesion layers supported by materials such as glass while maintaining their activity. In contrast, proteins must remain hydrated and kept at ambient temperatures, and are very sensitive to the physical and chemical properties of the support materials. Therefore, maintaining protein activity at the liquid-solid interface requires entirely different immobilization strategies than those used for nucleic acids. Additionally, the proper orientation of the protein at the interface is desirable to ensure accessibility of its active site(s) with interacting molecules. The detection/visualization methods commonly used in protein detection methods (e.g., enhanced chemiluminescence, ELISA) are generally not compatible with methods and apparatuses utilized by the cDNA microarray industry.

The need to detect multiplexed protein levels has generated considerable interest in developing a protein array analog to the highly successful cDNA microarray. The first step in developing protein microarrays is generally adapting existing cDNA microarray technology to the behavioral peculiarities of proteins at surfaces and protein affinity binding. Automatic and precise robotic printing and the commercially available fluorescence-detecting scanner systems can be taken advantage of directly; however, this requires several distinct modifications to the same basic assay format, particularly array production reagents and systems. Such modifications are not currently available in the art. Furthermore, there is no simple method of protein amplification, such as PCR amplification of DNA, so alternative suitable approaches for detecting very low levels of proteins must be achieved.

An adaptation of cDNA microarray methodologies to the stringent requirements of protein detection would be beneficial in the development of methods for the practical evaluation of wound healing-associated and biocompatibility-associated cytokines and growth factors.

### SUMMARY

A method of establishing a profile of one of host organism response to foreign, implanted material, wound healing, and both host organism response to foreign, implanted material and wound healing is disclosed. In some embodiments the method comprises: collecting a biological sample selected from the group consisting of (i) fluid from interstitial space between an implanted biomaterial and host organism tissue, (ii) supernatant from a cell culture to which biomaterial has been exposed, and (iii) a wound; contacting the biological sample with at least one microarray for the detection of cytokines and growth factors associated with one of host organism response to foreign, implanted material, wound healing, and both host organism response to foreign, implanted material and wound healing, the microarray comprising a plurality of capture antibody samples immobilized on a solid substrate to form a plurality of array elements, wherein: (i) each capture antibody sample comprises an anti-cytokine or an anti-growth factor capture antibody in a printing buffer solution; and (ii) each anti-cytokine or an anti-growth factor capture antibody specifically binds a cytokine or growth factor associated with one of host organism response to foreign, implanted material, wound healing, and both host organism response to foreign, implanted material and wound healing; detecting binding to the microarray of at least one cytokine or growth factor associated with one of host organism response to foreign, implanted material, wound healing, and both host organism response to foreign, implanted material and wound healing, wherein the binding indicates the presence in the biological sample of a cytokine or growth factor associated with one of host organism response to foreign, implanted material, wound healing, and both host organism response to foreign, implanted material and wound healing; and establishing a profile of one of host organism response to foreign, implanted material, wound healing, and both host organism response to foreign, implanted material and wound healing based on the binding.

A kit for establishing a profile of one of host organism response to foreign, implanted material, wound healing, and both host organism response to foreign, implanted material and wound healing is also disclosed. In some embodiments the method comprises: (a) at least one microarray for the detection of cytokines and growth factors associated with one of host organism response to foreign, implanted material, wound healing, and both host organism response to foreign, implanted material and wound healing, wherein the microarray comprises a plurality of capture antibody samples immobilized on a solid substrate to form a plurality of array elements, wherein: (i) each capture antibody sample comprises an anti-cytokine or an anti-growth factor capture antibody in a printing buffer solution; and (ii) each anti-cytokine or an anti-growth factor capture antibody specifically binds a cytokine or growth factor associated with one of host organism response to foreign, implanted material, wound healing, and both host organism response to foreign, implanted material and wound healing; (b) at least one reagent useful for the detection of cytokines and growth factors associated with one of host organism response to foreign, implanted material, wound healing, and both host organism response to foreign, implanted material and wound healing; and (c) instructions for establishing a profile of one of host organism response to foreign, implanted material, wound healing, and both host organism response to foreign, implanted material and wound healing based on the binding.

Also disclosed herein are cytokine detection arrays that adapt the cDNA microarray technology format to protein detection. Additionally disclosed and provided are methods designed specifically for fabricating the cytokine detection arrays and carrying out assays for the detection of cytokines and growth factors associated with wound healing and the host-organism response to implanted materials (i.e., biocompatibility). These methods utilize specifically designed array printing buffers and blocking buffers that have also been optimized for the sensitive detection of wound-healing and biocompatibility associated cytokines and growth factors. The low detection limit (about 10 pg/ml for cytokines or growth factors) and a broad linear dynamic range (over 4 orders of magnitude) of the optimized arrays permit the development of standard curves for assaying biological samples. The design advantageously overcomes the low reproducibility that frequently hinders microarray-based technology.

In some embodiments, a plurality of capture antibodies that are each specific for a cytokine or growth factor are bound to the surface of a solid substrate such as a slide or a “chip”. The capture antibodies can be printed in an array by robotically spotting the antibodies onto the substrate. In some embodiments, the concentration of capture antibody in each spot can range from about 250 μg/mL to about 500 μg/mL, while the spot itself (i.e., the total sample size) can have a diameter of about 160 μm and a volume of about 1.0 nL, although greater or lower concentrations of capture antibody and larger or smaller diameters and volumes for spots are also encompassed by the presently disclosed subject matter. Antibodies specific for numerous cytokines/growth factors can be spotted onto a slide at one time, which cytokines/growth factors include TGF-β1, TGF-β2, TGF-β3, PDGF, TNF-α, IL-6, VEGF, basic FGF, MCP-1, MIP-1α, IL-4, IL-8, IL-10, EGF, IGF-I, MIP-1β, IL-1ra, IL-13, IL-2, and others. Suitable solid substrates include modified glass slides (for example, nitrocellulose-coated glass slides), although other substrates can easily be used. The capture antibodies are spotted onto the solid substrate in a printing buffer, which in some embodiments comprises about 70% PBS and 30% glycerol/EDTA.

After the capture antibodies are spotted onto the substrate in an array, the substrate is blocked with a blocking buffer, which in some embodiments comprises 5% sucrose and 3% Tween 20. The solid substrate is then incubated with a test sample (e.g., a serum or bodily fluid sample) that contains or is suspected to contain cytokines and/or growth factors. The presence of cytokines and growth factors is detected on the solid substrate by, in some embodiments, an immunoassay, such as a sandwich immunoassay or a direct labeling assay. In some embodiments, biotin-conjugated detection antibodies that are also specific for the cytokines and growth factors are incubated with the array and then detected by streptavidin-conjugated fluorescent dyes. Provided herein are assay parameters (e.g., buffer component concentrations, dye concentrations, incubation times, and intermediary wash steps) that are optimal for the detection requirements of cytokines and/or growth factors related to wound healing and host organism responses to bioimplantation. The dynamic range of the assay provided by this method is around four orders of magnitude, while the sensitivity of the assay can be expressed in terms of a detection limit of about 10 pg/mL.

The microarrays and assay methods associated with the microarrays provide several particular method embodiments. One embodiment is a method for temporal profiling of molecular signaling in wound healing. In this aspect, fluid taken from a wound can be incubated with the array under the assay conditions of the presently disclosed subject matter; cytokines/growth factors present in the sample can be detected in picogram quantities.

Another embodiment is a method for determining the biocompatibility of biomaterial implants. Undesirable host responses (e.g., toxicity) can be indicated by the presence of cytokines and growth factors detectable by the arrays and assays of the presently disclosed subject matter. In such an embodiment, the putative material is exposed to, for example, cultured monocytes and/or macrophages, and the supernatant tested for the presence of cytokines/growth factors at different time points.

The presently disclosed subject matter includes one or more of several features that provide the user with significant advantages over cytokine/growth factor detection methods known in the art. These features include the quantitative parameters of the disclosed microarrays and assays that provide the user with the ability to test very small volumes of test material, to use very low quantities of capture antibodies, and to achieve very sensitive detection levels (e.g., detection of very low quantities of cytokines and/or growth factors present in the test sample). The ability to use sandwich immunoassay technology in combination with microarray technology also provides the user with the ability to make quantitative (as opposed to merely relative) measurements of cytokine/growth factor levels in test samples.

Another feature is the combination of protein array technology and microdialysis, which provides the user with the ability to test samples other than serum (e.g., bodily fluids from a wound site or a bioimplant-host interface obtained with a microdialysis probe).

Still another feature is the ability to detect extremely low levels of target cytokines/growth factors; e.g., in picogram quantities. In this regard, the present embodiments provide advantages over known methods of specific protein detection, which include Western blots, ELISA, and mass spectroscopy.

The sensitivity provided by the present embodiments is particularly significant in light of the study of cytokines and growth factors under physiological conditions. With regard to studying wound healing and the production of such molecules during a host response to a bioimplant, tissue cytokine levels must be detected in picogram quantities of short-lived species. Additionally, when detecting cytokines produced in a host response to a bioimplant, the cytokine molecules will be resident in interstitial volumes that are extremely small and almost inaccessible. The ability to assay very small volumes of sample that might contain very low levels of cytokine/growth factor is a feature of one ore more of the embodiments disclosed herein.

### BRIEF DESCRIPTION OF THE DRAWINGS

FIG. 1 is a graphical plot illustrating the identification of anti-human TNF-α antibody concentrations. Six identical arrays on a single slide consisting of a series of TNF-α capture antibodies at varying concentrations were incubated with five different concentrated target TNF-α samples and one high-concentration cytokine cocktail without TNF-α. Based upon these illustrated results, a concentration of 250-500 μg/ml capture antibody was found to be a useful concentration range for the TNF-α cytokine-specific protein microarray. This concentration range provided for the detection of low concentrations of cytokine (e.g., 10 pg/ml TNF-α) with no non-specific cross reactivity in a multiplex assay

FIG. 2 shows visualized microarrays illustrating that non-specific cross reactivity was greatly reduced or eliminated by the use of optimized cytokine detection protein arrays. Six identical arrays on a single slide were simultaneously exposed to 100 ng/ml IL-1β (A), TNF-α (B), VEGF (C), MIP-1β (D), TGF-β1 (E), as well as a cocktail of all five cytokines (F). Incubation and detection were carried out as disclosed in the Examples. From A to E, all binding occurred only at the specific capture antibody sites. F was used as a control to confirm the validity of all the tested cytokines and the effectiveness of the procedure.

FIG. 3 shows visualized microarrays illustrating the detection of five cytokines in a dose-response format. Six identical arrays on a single slide were simultaneously exposed to cocktails of five cytokines at concentration of 100 ng/ml (A), 10 ng/ml (B), 1 ng/ml (C), 100 pg/ml (D), 10 pg/ml (E), and diluent only (F). Incubation and detection were carried out as disclosed in the Examples. From A to E, in the presence of target cytokines, signals were detected for all cytokines. With the concentrations of the cytokine cocktail decreased, the corresponding cytokine signals decreased. For F, in the absence of cytokines only the detection control localized apparent signal.

FIG. 4 is a sigmoid curve typical of dose responses for individual cytokines assayed in multiplex. A: IL-1β, B: TNF-α, C: VEGF, D: MIP-1β, E: TGF-β1. The corresponding data for the relevant standard curves are listed in Table 5. All the fluorescent intensities in this plot refer to background subtracted fluorescent intensities.

FIGS. 5A, 5B, and 5C illustrate three applications of optimized cytokine-detecting microarrays.

FIG. 5A shows a visualized microarray illustrating an array response to a solution into which VEGF is released from a hydrogel. VEGF was detected and was 9.08±0.35 ng/ml.

FIG. 5B shows a visualized microarray illustrating an array response to patient serum #1. VEGF and TGF-β1 were detected, and were respectively 133±36 pg/ml and less than 10 pg/ml.

FIG. 5C shows a visualized microarray illustrating an array response to patient serum #2. VEGF, MIP-1β, and TGF-β1 were detected, and were respectively 600±100 pg/ml, 15 ±5 pg/ml, and less than 10 pg/ml.

FIG. 6 is a schematic diagram illustrating a two-chamber configuration in which direct label and sandwich immunoassays can be carried out on the same slide.

FIG. 7 is a visualized protein microarray showing direct label and sandwich immunoassay images scanned on the same slide.

FIG. 8 is a series of bar graphs illustrating cytokine signals for the direct label and sandwich assays on four different slides. A: IL-1β, B: TNF-α, C: VEGF, D: MIP-1β, E: TGF-β1. The number adjacent to each column is a ratio of background subtracted fluorescent intensity of sandwich assay to background subtracted fluorescent intensity of direct label assay.

FIG. 9 depicts the overall strategy for using the microarrays disclosed herein in three representative, non-limiting applications.

FIGS. 10A-10C depict cytokine expression patterns using an 8×5 array.

FIG. 10A depicts a profile induced by bacterial lipopolysaccharide (LPS), FIG. 10B depicts a profile induced by titanium (Ti) particles, and FIG. 10C depicts a negative control (no treatment) profile.

FIGS. 11A-11C depict relationships between cell-material interaction time and concentration of four cytokines (IL-6, TNF-α, MIP-2, and TGF-β1) secreted by monocytes in culture.

FIG. 11A depicts a 72 hour time course of expression of the four cytokines in response to LPS exposure. FIG. 11B depicts a 72 hour time course of expression of the four cytokines in response to exposure to Ti particles. FIG. 11C depicts a 72 hour time course of expression of the four cytokines in a negative control (no treatment).

### DETAILED DESCRIPTION

The presently disclosed subject matter will be now be disclosed more fully hereinafter with reference to the accompanying Examples, in which representative embodiments of the presently disclosed subject matter are shown. The presently disclosed subject matter can, however, be embodied in different forms and should not be construed as limited to the embodiments set forth herein. Rather, these embodiments are provided so that this disclosure will be thorough and complete, and will fully convey the scope of the presently disclosed subject matter to those skilled in the art.

Unless otherwise defined, all technical and scientific terms used herein have the same meaning as commonly understood by one of ordinary skill in the art to which the presently disclosed subject matter belongs. All publications, including patent applications, patents, scientific literature, and other references mentioned herein are incorporated by reference in their entireties.

Throughout the specification and claims, a given chemical formula or name shall encompass all optical and stereoisomers as well as racemic mixtures where such isomers and mixtures exist.

Many biomaterial implants, even those considered to be highly successful, fail after long-term implantation. For example, more than 500,000 artificial hips and knees implanted each year in the United States, but only very few survive 25 years. Virtually all of these late onset failures can be traced to aseptic loosening arising from chronic and adverse reactions of inflammatory leukocytes to wear generated debris. This mode of frustrated or incomplete healing of the implant wound site is a form of bioincompatibility unrelated to cytotoxicity that cannot be detected using standard cytotoxicity test. A more sensitive biomaterial screen test is needed for selecting or engineering more biocompatible implants, and is provided in accordance with some embodiments of the presently disclosed subject matter.

Monocytes/macrophages are the sentinel cells that direct the inflammation and wound healing responses to implants via expression patterns of secreted cytokines and growth factors. Also disclosed herein are methods for establishing a temporal profile of the cytokines and growth factors secreted in response to a test material, which thus creates a unique “biosignature” reflecting the material's potential biocompatibility. Thus, the terms “profile” and “biosignature” are used interchangeably herein and are meant to refer to the combinations of cytokines and growth factors secreted in response to one or more test materials, to one or more test conditions (e.g. the presence of a wound), or to any other stimulus as would be apparent to one of ordinary skill in the art after a review of the present disclosure.

In some embodiments, the presently disclosed methods employ an antibody array capable of detecting inflammation and wound healing related cytokines and growth factors, and a profile, or biosignature, for a test material is established. Representative embodiments of the presently disclosed methods include exposing cells indicative of the wound healing environment, including but not limited to monocytes/macrophages, fibroblasts, and endothelial cells, in culture to a test biomaterial, and use the high throughput protein microarrays to determine, in parallel, the temporal profile of cytokines and growth factors released from the cells interrogating the biomaterial. Microarray software can also provided to interpret protein array results and patterns of cytokines or growth factors that are markers for biocompatibility or bioincompatibility are identified and optionally maintained in a database for use in comparisons or for otherwise evaluating choices of particular implant materials in a given subject.

Microarrays disclosed herein are protein detection microarrays and are useful in the parallel detection of cytokines and growth factors. Cytokines and growth factors detected by the presently disclosed microarrays are those cytokines and growth factors associated with wound healing and host organism responses to the implantation of foreign materials (e.g., biomaterials and sensors). By “associated with” is meant that the cytokine and/or growth factor is either produced in response to the wound healing or material implanting event, or is involved in a wound healing or host organism response signaling pathway. These cytokines and growth factors include, but are not limited to, TNF-α, basic FGF, PDGF, VEGF, MIP-1, IL-1β, TGF-β1, TGF-β2, TGF-β3, G-CSF, IL-10, GM-CSF, IL-13, GROα, IL-15, IFN-γ, MCP-1, IL-1α, MCP-2, IL-2, MCP-3, IL-3, MIG, IL-5, TGF β1, IL-6, IL-7, IL-8, TNF-β, IL-12, IL-11, MIP-1β, sICAM-1, IL4, IL-5, IFN-α, SAA, IL-13, sVCAM-1, APO-1, GM-CSF, and IL-16. In certain embodiments, the microarrays specifically detect basic-FGF, PDGF, TNF-α, TGF-α, IL-1α, IL4, IL-6, IL-8, and IL-10, all of which are macrophage-derived mediators of wound healing and inflammation.

Microarrays disclosed herein are solid phase arrays comprising a plurality of different antibodies arrayed in corresponding discrete array elements and specific for a corresponding plurality of different cytokines. As used herein, an “array” or a “microarray” is an ordered arrangement of proteins, particularly antibodies, located in addressable locations on a solid substrate. The array elements are arranged so that there are in some embodiments at least one or more different array elements, in some embodiments at least 10 array elements, in some embodiments at least 100 array elements, and in some embodiments 1,000 to 10,000 array elements on a 1 cm² substrate surface. Each array element is a discrete sample of one antibody that has been immobilized onto the surface of a solid substrate, as these terms are further defined herein. Array elements are also referred to herein, interchangeably, as “spots” or “patches”.

Within an array, each array element is addressable, meaning that its location can be reliably and consistently determined within the dimensions of the array surface. Thus, in ordered arrays the location of each discrete antibody element is assigned to the element at the time when it is spotted onto the array surface. Usually, a key is provided in order to correlate each location with the appropriate target. Ordered arrays are generally arranged in a symmetrical grid pattern, but elements can optionally be arranged in other patterns (e.g., in radially distributed lines or ordered clusters).

In particular embodiments, microarrays comprise at least five different antibodies arrayed in corresponding discrete array elements and specific for corresponding five different cytokines. In other embodiments, microarrays comprise at least ten different antibodies arrayed in corresponding discrete array elements and specific for corresponding at least ten different cytokines. In some embodiments, microarrays comprise at least fifteen different antibodies arrayed in corresponding discrete array elements and specific for corresponding at least fifteen different cytokines. The array elements are generally discrete regions of a substrate surface in fluid connection such that all the elements of the array can be incubated, washed, etc., in a single continuous medium. Hence, microarrays as disclosed herein are distinct from assay formats where each specific antibody is separated in discrete, fluid-separated incubation wells, such as in a microtiter plate.

Microarrays can be made according to techniques that are disclosed in the present disclosure. In some embodiments, anti-cytokine and or anti-growth factor capture antibodies are immobilized on a solid support such that a position on the support identifies a particular and pre-selected set of capture antibodies.

As used herein, the term “antibody” means intact immunoglobulin molecules, chimeric immunoglobulin molecules, or antibody fragments, whether natural or wholly or partially synthetically produced. All derivatives thereof that maintain specific binding ability are also included in the term. The term also covers any protein having a binding domain that is homologous or largely homologous to an immunoglobulin binding domain. These proteins can be derived from natural sources, or partly or wholly synthetically produced. An antibody can be monoclonal or polyclonal. The antibody can be a member of any immunoglobulin class, including any of the human classes: IgG, IgM, IgA, IgD, and IgE. Derivatives of the IgG class are particular embodiments of the presently disclosed subject matter.

The term “antibody fragment” refers to any derivative of an antibody that is less than full-length. In some embodiments, the antibody fragment retains at least a significant portion of the full-length antibody's specific binding ability. Examples of antibody fragments include, but are not limited to, Fab, Fab′, F(ab′)₂, scFv, Fv, and Fd fragments. The antibody fragment can be produced by approaches known in the art. For instance, the antibody fragment can be enzymatically or chemically produced by fragmentation of an intact antibody or it can be recombinantly produced from a gene encoding the partial antibody sequence. Alternatively, the antibody fragment can be wholly or partially synthetically produced. The antibody fragment can optionally be a single chain antibody fragment. Alternatively, the fragment can comprise multiple chains that are linked together, for instance, by disulfide linkages. The fragment can also optionally be a multimolecular complex. A functional antibody fragment will typically comprise at least about 50 amino acids and more typically will comprise at least about 200 amino acids.

The term “bind”, as used herein, refers to the well understood antigen/antibody binding as well as to other nonrandom associations between an antigen and an antibody. The term “specifically bind”, as used herein describes an antibody or other ligand that does not cross react substantially with any antigen other than the antigen, or antigens, specified.

Antibodies and antibody fragments can be produced by techniques well known in the art, which include those disclosed in Harlow and Lane, Antibodies: A Laboratory Manual, Cold Spring Harbor Laboratory Press, Cold Spring Harbor, New York, United States of America (1989); Kohler et al., Nature 256, 495-97 (1975) and U.S. Pat. Nos. 5,545,806; 5,569,825; and 5,625,126, each of which is incorporated herein by reference. Antibodies, as defined herein, also include single chain antibodies (scFv) comprising linked V_{H} and V_{L} domains that retain the conformation and specific binding activity of the native idiotype of the antibody. Such single chain antibodies can be produced by methods known in the art. See e.g., Alvarez et al., Human Gene Therapy 8, 229-242 (1997).

In producing antibodies for use in the presently disclosed microarrays and methods, suitable cytokines can be purchased in purified or recombinant form from commercial sources, expressed from commercially and/or publicly available clones, and/or purified from tissues. In some embodiments, the cytokines are of native human sequence, although homologs from a wide variety of animal species, particularly mammalian (e.g., murine) species, are frequently available and can be used.

The term “capture antibody”, as used herein, is an immobilized antibody that binds, is bound by, or forms a complex with, one or more cytokines and/or growth factors of interest in a sample to be tested. Capture antibodies, as used herein, specifically bind cytokines and growth factors that are associated with wound healing and host organism response to the implantation of foreign materials. Suitable capture antibodies include, but are not limited to, anti-cytokine antibodies including, but not limited to, anti-human G-CSF, anti-human IL-10, anti-human GM-CSF, anti-human IL-13, anti-human GROα, anti-human IL-15, anti-human IFN-γ, anti-human MCP-1, anti-human IL-1α, anti-human IL-1β, anti-human IL-1ra, anti-human MCP-2, anti-human IL-2, biotinylated anti-human MCP-3, anti-human IL-3, anti-human MIG, anti-human IL-5, anti-human/mouse/pig TGF-β1, anti-human/mouse/pig TGF-β2, anti-human/mouse/pig TGF-β3, anti-human PDGF-BB, anti-human VEGF, anti-human basic FGF, anti-human EGF, anti-human IGF-I, anti-human IL-6, anti-human RANTES, anti-human IL-7, anti-human TNF-α, anti-human IL-8, anti-human TNF-β, anti-human ENA-78 antibody, anti-human 1-309 antibody, anti-human IL-11 antibody, anti-human IL-12 antibody, anti-human IL-15 antibody, anti-human IL-17 antibody, anti-human M-CSF antibody, anti-human MDC antibody, anti-human MIP-1α antibody, anti-human MIP-1β antibody, anti-human MIP-1γ/Leukotactin antibody, anti-human SCF antibody, anti-human/mouse SDF-1 antibody, and anti-human IL-4 antibody. In some embodiments, suitable capture antibodies include, but are by no means limited to anti-human TGF-β1, anti-human TGF-β2, anti-human TGF-β3, anti-human PDGF-BB, anti-human IL-1β, anti-human IL-1ra, anti-human TNF-α, anti-human IL-6, anti-human VEGF, anti-human basic FGF, anti-human MCP-1, anti-human MIP-1α, anti-human IL-4, anti-human IL-8, anti-human IL-10, anti-human EGF, and anti-human IGF-I. In some embodiments, suitable capture antibodies include, but are by no means limited to, anti-human TNF-α IgG; anti-rat IL-1 IgG; anti-rat IL-6 IgG; anti-human IL-8 IgG; anti-rat IL-10 IgG; and anti-mouse IL-12 IgG.

As is known in the art, antibody preparations can be either polyclonal or monoclonal. In some embodiments, the immobilized capture antibodies are monoclonal antibodies. The term “monoclonal antibody” refers to a population of antibody molecules that contain only one species of paratope and thus typically display a single binding affinity for any particular epitope with which it immunoreacts; a monoclonal antibody can have a plurality of antibody combining sites, each immunospecific for a different epitope, e.g., a bispecific monoclonal antibody. Methods of producing a monoclonal antibody, a hybridoma cell, or a hybridoma cell culture are known in the art.

Numerous monoclonal antibodies are available commercially. Monoclonal antibodies can alternatively be obtained by methods known to those skilled in the art. The production of monoclonal antibodies against specific protein targets is routine using standard hybridoma technology. They can be obtained by any technique that provides for the production of antibody molecules by continuous cell lines in culture. See e.g., Kohler et al., Nature 256, 495-497 (1975); Kohler et al., Eur. J. Immunol. 6, 511 (1976); Kohler et al., Eur. J. Immunol. 6, 292 (1976); Hammerling et al., in: Monoclonal Antibodies and T-Cell Hybridomas (Elsevier, New York, N.Y., United States of America (1981) pp. 563-681), Harlow and Lane Antibodies: A Laboratory Manual (Cold Spring Harbor Laboratory Press, Cold Spring Harbor, N.Y., United States of America (1988)) and U.S. Pat. No. 4,376,110.

Antibodies, either polyclonal, monoclonal, or derivatives thereof (e.g. single chain fragment variable (scFv) antibodies, humanized antibodies, etc.) can be labeled with a detectable moiety. As used herein, the phrase “detectable moiety” refers to a chemical group that can be attached to an antibody or antibody derivative that allows for the detection of the antibody. Representative detectable moieties include covalently attached chromophores, fluorescent moieties, enzymes, antigens, groups with specific reactivity, chemiluminescent moieties, and electrochemically detectable moieties, etc. In some embodiments, a detectable moiety is a biotin molecule, which can be detected based upon its interaction with avidin or streptavidin. In some embodiments, the antibodies are biotinylated. In some embodiments, the biotinylated antibodies are detected using a secondary antibody that comprises an avidin or streptavidin group and is also conjugated to a fluorescent label including, but not limited to Cy3 and Cy5. Additional detection strategies are described hereinbelow.

The solid substrates on which capture antibodies are immobilized in an array are generally planar (i.e., two-dimensional), although three-dimensional substrates can optionally be used. The solid substrates are in some embodiments substantially rigid and amenable to capture antibody immobilization and detection methods. In the case of fluorescent detection, the substrate has low background fluorescence in the region of the fluorescent dye excitation wavelengths. As used herein, solid substrates are compatible with existing microscope slide based systems, and are microporous in order to absorb and hold anti-cytokine capture antibodies.

Representative examples of solid supports include, but are not limited to nitrocellulose; glass; silica; silica gel; silicon wafer; silicone; plastics such as those made of polyethylene, polystyrene, polyvinyl chloride (PVC), or polyvinyl pyrrolidone (PVP); nylon; TEFLON®; nitrocellulose; ceramic; fiber optic; and semiconductor material. The substrate can also be a combination of any of the aforementioned substrate materials. A non-limiting example of such a combination substrate is a modified glass slide, for example, a glass slide coated with a nitrocellulose layer or a nitrocellulose polymer.

When using a glass substrate, the glass should be substantially free of debris and other deposits and have a substantially uniform coating. Pretreatment of slides to remove organic compounds that can be deposited during their manufacture can be accomplished, for example, by washing in hot nitric acid. Cleaned slides can optionally be coated with 3-aminopropyltrimethoxysilane using vapor-phase techniques. After silane deposition, slides are washed with deionized water to remove any silane that is not attached to the glass and to catalyze unreacted methoxy groups to cross-link to neighboring silane moieties on the slide. The uniformity of the coating can be assessed by known methods, for example electron spectroscopy for chemical analysis (ESCA) or ellipsometry (Ratner & Castner (1997) in Vickerman, ed., Surface Analysis: The Principal Techniques, John Wiley & Sons, New York, N.Y., United States of America; Schena et al. (1995) Science 270:467-470). See also Worley et al. (2000) in Schena, ed., Microarray Biochip Technology, pp. 65-86, Eaton Publishing, Natick, Mass., United States of America.

In some embodiments, modified glass slides, such as but not limited to nitrocellulose-coated glass slides, are used to produce optimized cytokine detection protein arrays. An example of a suitable solid substrate is a FAST® slide, available from Schleicher & Schuell (Keene, N.H., United States of America).

A microarray for the detection of cytokines and/or growth factors in a biological sample can be constructed using any one of several methods available in the art, including but not limited to photolithographic and microfluidic methods. In some embodiments, contact printing using rigid pins is used to produce the cytokine/growth factor-specific microarray. In some embodiments, the cytokine/growth factor-specific microarray is produced by robotic contact printing.

Several procedures and tools have been developed for printing microarrays using rigid pin tools, and these can be used in accordance with the subject matter disclosed herein. In surface contact printing, the pin tools are dipped into a solution of, for example, capture antibody, resulting in the transfer of a small volume of fluid onto the tip of the pins. Touching the pins or pin samples onto a microarray solid substrate surface leaves a spot, the diameter of which is determined by the surface energies of the pin, fluid, and microarray surface. Typically, the transferred fluid comprises a volume in the nanoliter or picoliter range.

One common contact printing technique uses a solid pin replicator. A replicator pin is a tool for picking up a sample from one stationary location and transporting it to a defined location on a solid support. A typical configuration for a replicating head is an array of solid pins, generally in an 8×12 format, spaced at 9-mm centers that are compatible with 96- and 384-well plates. The pins are dipped into the wells, lifted, moved to a position over the microarray substrate, and lowered to touch the solid support, whereby the sample is transferred. The process is repeated to complete transfer of all the samples. See Maier et al. (1994) J Biotechnol 35:191-203. A recent modification of solid pins involves the use of solid pin tips having concave bottoms, which print more efficiently than flat pins in some circumstances. See Rose (2000) in Schena, ed., Microarray Biochip Technology, pp. 19-38, Eaton Publishing, Natick, Mass., United States of America. Other formats, such as but not limited to 8×5 formats, can also be employed.

Solid pins for microarray printing can be purchased in a wide range of tip dimensions, for example, from TeleChem International, Inc. of Sunnyvale, Calif., United States of America. The CHIPMAKER™ and STEALTH™ pins from TeleChem contain a stainless steel shaft with a fine point. A narrow gap is machined into the point to serve as a reservoir for sample loading and spotting. The pins have a loading volume of 0.2 μl to 0.6 μl to create spot sizes ranging from 75 μm to 360 μm in diameter.

To permit the printing of multiple arrays with a single sample loading, quill-based array tools, including printing capillaries, tweezers, and split pins, have been developed. These printing tools hold larger sample volumes than solid pins and therefore allow the printing of multiple arrays following a single sample loading. Quill-based arrayers withdraw a small volume of fluid into a depositing device from a microwell plate by capillary action. See Schena et al. (1995) Science 270:467-470. The diameter of the capillary typically ranges from about 10 μm to about 100 μm. A robot then moves the head with quills to the desired location for dispensing. The quill carries the sample to all spotting locations, where a fraction of the sample is deposited at each location. The forces acting on the fluid held in the quill must be overcome for the fluid to be released. Accelerating and then decelerating by impacting the quill on a microarray substrate accomplishes fluid release. When the tip of the quill contacts the solid support, the meniscus is extended beyond the tip and transferred onto the substrate. Carrying a large volume of sample fluid minimizes spotting variability between arrays.

In the presently disclosed methods, a solid replicating pin is dipped in a capture antibody solution comprising a capture antibody specific for a cytokine or growth factor in a printing buffer. In some embodiments, the printing buffer comprises about 70% phosphate buffered saline (PBS) and 30% glycerol/EDTA. The concentration of capture antibody in the printing buffer solution can range in some embodiments from about 250 μg/mL to about 500 μg/mL, although lower and higher concentrations of capture antibodies can also be employed. After the pin is dipped into the solution, the liquid on the tip of the pin is transferred and deposited onto the surface of the solid substrate.

In the practice of the presently disclosed methods, the volume deposited per spot is in some embodiments about 10 pL to about 10 nL, and in some embodiments about 500 pL to about 2.0 nL. In some embodiments, the volume of capture antibody transferred to the surface of the solid substrate is less than about 5.0 nL. In some embodiments, the volume of capture antibody transferred to the surface of the solid substrate is about 1.0 nL. The diameter of each spot is in some embodiments about 50 μm to about 1000 μm, and in some embodiments about 100 μm to about 250 μm. In particular embodiments, the dipping and transferring of the capture antibody is carried out robotically, and/or is carried out in multiplex and parallel format. Array printing can be performed at relatively high humidity (about 70%). Newly printed arrays should be dried in a manner that allows antibodies to bind fully to the substrate surface, in general from about one to about three hours. The dried arrays can be blocked with a suitable buffer for immediate use, as disclosed further herein, or stored in PBS or a desiccator at 2-8° C.

As is standard in the art, a printing technique for making a microarray creates consistent and reproducible spots. Each spot is preferably uniform, and appropriately spaced away from other spots within the array configuration. The elements of the array can be of any geometric shape. For instance, the elements can be rectangular or circular. The elements of the array can also be irregularly shaped. The distance separating the elements of the array can vary. In some embodiments, the elements of the array are separated from neighboring patches by about 1 μm to about 500 μm.

In certain embodiments, each element is circular in shape and has a diameter of from about 50 μm to about 250 μm. In particular embodiments, each element has a diameter of about 150 μm to about 180 μm. In a specific embodiment, each element has a diameter of about 160 μm.

Immobilized capture antibodies can be associated with the solid substrate by covalent bonds and/or via non-covalent attractive forces such as hydrogen bond interactions, hydrophobic attractive forces, and ionic forces, for example. In some embodiments, the capture antibodies are non-covalently attached to the surface of the solid substrate.

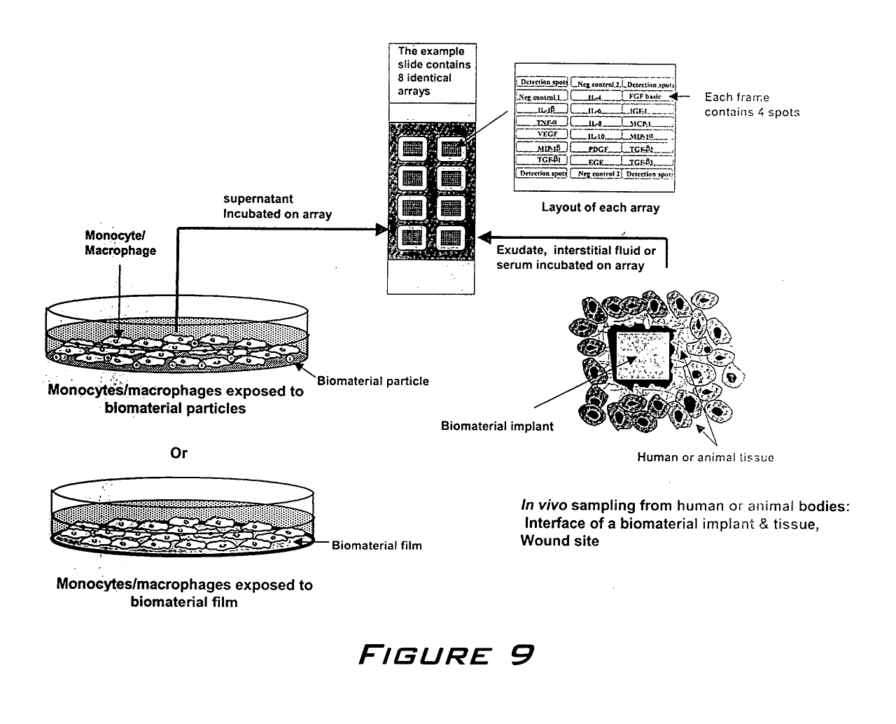

Microarrays produced according to the foregoing methods can be used in methods of detecting cytokines and growth factors associated with wound healing and host organism responses to implantation of foreign materials (the latter use is depicted generally in FIG. 9). Thus, also disclosed herein are methods for establishing a temporal profile of the cytokines and growth factors secreted in response to a test material, which thus creates a unique “biosignature” reflecting the material's potential biocompatibility. Thus, the terms “profile” and “biosignature” are used interchangeably herein and are meant to refer to the combinations of cytokines and growth factors expressed in response to one or more test materials, to one or more test conditions (e.g. the presence of a wound), or to any other stimulus as would be apparent to one of ordinary skill in the art after a review of the present disclosure. Apoptosis level of macrophages on the surface of biomaterials can also be used in establishing a profile.

In some embodiments, the presently disclosed methods employ an antibody array capable of detecting inflammation and wound healing related cytokines and growth factors, and a profile, or biosignature, for a test material is established. Representative embodiments of the presently disclosed methods include exposing cells indicative of the wound healing environment, including but not limited to monocytes/macrophages, fibroblasts, and endothelial cells, in culture to a test biomaterial, and use the high throughput protein microarrays to determine, in parallel, the temporal profile of cytokines and growth factors released from the cells interrogating the biomaterial. Microarray software can also provided to interpret protein array results and patterns of cytokines or growth factors that are markers for biocompatibility or bioincompatibility are identified and optionally maintained in a database for use in comparisons or for otherwise evaluating choices of particular implant materials in a given subject.

In some embodiments, methods of establishing a profile of one of host organism response to foreign, implanted material, wound healing, and both host organism response to foreign, implanted material and wound healing are provided. In some embodiments the methods comprise:
collecting a biological sample selected from the group consisting of (a) fluid from interstitial space between an implanted biomaterial and host organism tissue, (b) supernatant from a cell culture to which biomaterial has been exposed, and (c) a wound;
contacting the biological sample with at least one microarray for the detection of cytokines and growth factors associated with one of host organism response to foreign, implanted material, wound healing, and both host organism response to foreign, implanted material and wound healing, the microarray comprising a plurality of capture antibody samples immobilized on a solid substrate to form a plurality of array elements, wherein:
(i) each capture antibody sample comprises an anti-cytokine or an anti-growth factor capture antibody in a printing buffer solution; and
(ii) each anti-cytokine or an anti-growth factor capture antibody specifically binds a cytokine or growth factor associated with one of host organism response to foreign, implanted material, wound healing, and both host organism response to foreign, implanted material and wound healing;
detecting binding to the microarray of at least one cytokine or growth factor associated with one of host organism response to foreign, implanted material, wound healing, and both host organism response to foreign, implanted material and wound healing, wherein the binding indicates the presence in the biological sample of a cytokine or growth factor associated with one of host organism response to foreign, implanted material, wound healing, and both host organism response to foreign, implanted material and wound healing; and
establishing a profile of one of host organism response to foreign, implanted material, wound healing, and both host organism response to foreign, implanted material and wound healing based on the binding.

The terms “bio-compatible” and “biomaterial” are used interchangeably herein and are meant to refer to a material that is compatible with a biological system, yet is also foreign to the biological system. Thus, the terms “bio-compatible” and “biomaterial” can refer to a material that can be implanted internally in a subject as described herein.

Representative medical device biomaterial structures including but are not limited to the following: sensors (including but not limited to glucose sensors), pegs, stents, screws, nails, patches, tubes, plates, dressings, bandages, and/or another device that can be implanted in or otherwise provided to a subject in need thereof, as well as biomaterials that can be used to make such medical devices.

With respect to the methods of the presently disclosed subject matter, any animal subject can be a candidate for treatment. The term “subject” as used herein refers to any vertebrate species. The methods of the presently claimed subject matter are particularly useful in the diagnosis and treatment of warm-blooded vertebrates. Thus, the presently claimed subject matter concerns mammals. In some embodiments provided is the diagnosis and/or treatment of mammals such as humans, as well as those mammals of importance due to being endangered (such as Siberian tigers), of economical importance (animals raised on farms for consumption by humans) and/or social importance (animals kept as pets or in zoos) to humans, for instance, carnivores other than humans (such as cats and dogs), swine (pigs, hogs, and wild boars), ruminants (such as cattle, oxen, sheep, giraffes, deer, goats, bison, and camels), and horses. Also provided is the diagnosis and/or treatment of livestock, including, but not limited to domesticated swine (pigs and hogs), ruminants, horses, poultry, and the like.

The present microarrays and methodologies comprise a “biocompatibility array” for biomaterials screening, and methods to profile many biocompatibility related cytokines and growth factors and simultaneously measure their levels. The biocompatibility array is used to determine the profile of cytokines and growth factors released as a result of cells indicative of the wound healing environment interrogating the biomaterial surface. According to standard curves obtained at the same time on the same microarrays, information of cytokine and growth factor expression patterns and levels are provided. Furthermore, with reference to positive or negative controls, the biocompatibility of the tested biomaterials can be predicted. In addition, this array detects coordinating cytokines or growth factors on a specific biomaterial, which are used to further modify and/or optimize the potential biomaterials.

In general, delivery of biological samples solutions comprising cytokines and/or growth factors to be detected by the cytokine/growth factor-specific microarray is preceded or accompanied by delivery of a blocking buffer solution to the microarray. A blocking solution comprises a moiety that adheres to sites of non-specific binding on the array. For instance, solutions of bovine serum albumin or milk are commonly used as blocking solutions. Blocking buffer should have no intrinsic fluorescence and should form a layer that resists non-specific protein adhesion to the microarray. The inventors have surprisingly discovered that the optimal blocking buffer for cytokine/growth factor detection comprises 5% sucrose and 3% Tween 20.

In some embodiments, the biological sample is a body fluid. A “body fluid” can be any liquid substance extracted, excreted, or secreted from an organism or a tissue of an organism. The body fluid need not necessarily contain cells. Body fluids of relevance include, but are not limited to, whole blood, serum, urine, plasma, cerebral spinal fluid, tears, synovial fluid, and amniotic fluid.

In some embodiments, a biological sample is collected from a wound. The sample can be solid or liquid. Wound fluid is known to contain plasma, proteins, antibodies, red and white blood cells (erythrocytes and leukocytes), and platelets, although the presence or absence of any particular component is not required.

In some embodiments, a biological sample is collected from the interstitial space between an implanted material and a tissue in which the material is implanted. In certain embodiments, the biological sample is collected by microdialysis. In particular embodiments, the biological sample is collected by subcutaneous microdialysis. In some embodiments, a biological sample is supernatant produced by contacting putative or known bioimplantable material with cells indicative of the wound healing environment, including but not limited to monocytes/macrophages, fibroblasts, and endothelial cells, in a cell culture media.

Microdialysis probes are continuous perfusion devices designed for the site-specific microsampling and/or microperfusion of tissues. Probes comprise a single hollow fiber dialysis membrane placed at the distal tip of a bifurcated perfusion loop. Perfusate diverted from a central catheter flows up the lumen of the hollow fiber, allowing molecular exchange with the tissue space surrounding the probe. One can either deliver molecules from the perfusate to the surrounding tissue space (microperfusion), and/or analyze the perfusate collected distally from the probe tip for molecules collected from the surrounding tissue (microsampling). Microdialysis probes designed for brain and subcutaneous tissue are commercially available. The dimensions of a typical subcutaneous microdialysis probe tip are 10 mm in length and 0.5 mm in diameter.

In vivo microdialysis has been used to study cytokine-related activity in the brain, uterus, peritoneum, eye, skin, in wounded tissue, and in cell culture. See e.g., H. Anisman et al., Brain Research 731, 1-11 (1996); P. Licht et al., Seminars in Reproductive Medicine 19, 37-47 (2001); P. Jonsson, Gastroenterologia Japonica 27, 529-535 (1992); P. G. Osborne et al., Brain Research 661, 237-242 (1994); L. J. Petersen et al., Journal of Allergy and Clinical Immunology 98, 790-796 (1996); S. A. Brown et al. Plastic and Reconstructive Surgery 105, 991-998 (2000); and Y. S. Wu et al., Journal of Chromatography A 913, 341-347 (2001). The advent of immunoaffinity capillary electrophoresis (ICE) has pushed the detection limit of cytokine microdialysis sampling down to the fg/mL level. In ICE, collected microliter dialysate fractions are labeled fluorescently and injected into an immunoaffinity capillary that binds the specific cytokine, allowing other species to pass. Bound cytokines are then eluted electrophoretically and detected using laser-induced fluorescence (LIF). ICE/LIF has been employed to detect 10-2000 fg/mL of TNF-α in microdialysate samples collected from the wound site of a canine tibial fracture model. S. A. Brown et al. Plastic and Reconstructive Surgery 105, 991-998 (2000). More recently, ICE/LIF has been used to detect 100-200 fg/mL levels of IL-5, IL-5, and IL-10 secreted from a single CD4+ lymphocyte cell in vitro. T. M. Phillips, Luminescence 16, 145-152 (2001).

Use of the microarrays of the present disclosure can optionally involve placing the two-dimensional protein array in a flowchamber with approximately 1-10 microliters of fluid volume per 25 mm² overall surface area. The cover over the array in the flowchamber can be, for example, transparent or translucent, although this is not necessary to the proper practice of the presently disclosed subject matter. In some embodiments, the cover can comprise Pyrex or quartz glass. In other embodiments, the cover can be part of a detection system that monitors interaction between biological moieties immobilized on the array and an analyte. The flow chambers should remain filled with appropriate aqueous solutions to preserve protein activity. Salt, temperature, and other conditions can be kept similar to those of normal physiological conditions. Biological samples can be flushed into the flow chamber as desired and their interaction with the immobilized proteins determined. Sufficient time must be given to allow for binding between the immobilized antibodies and a cytokine and/or growth factor to occur. No specialized microfluidic pumps, valves, or mixing techniques are required for fluid delivery to the array.

In certain embodiments, biological samples in fluid form are delivered to each of the spots of the microarray individually. For instance, in some embodiments, the regions of the substrate surface can be microfabricated in such a way as to allow integration of the array with a number of fluid delivery channels oriented perpendicular to the array surface, each one of the delivery channels terminating at the site of an individual protein-coated patch.

The presence of cytokines and/or growth factors in a biological sample is detected on the microarray by use of an immunoassay. In some embodiments, the immunoassay is a direct labeling assay. In alternative embodiments, the immunoassay is a sandwich assay. In particular embodiments, both a direct labeling and a sandwich assay are carried out simultaneously on the same microarray.

Direct label assays provide an immediate analog to cDNA microarray technology, which generally is a competitive binding assay between a sample and reference labeled with different dyes, typically the fluorescent dyes Cy3 and Cy5. Direct labeling systems using Cy3 and Cy5 dyes are commercially available from BD Biosciences Clontech (Palo Alto, Calif., United States of America) and other manufacturers. The competitive direct label assay provides a relative measurement of the change in expression level of the various proteins. Unfortunately, the absolute intensity observed at a particular spot on a chip can be meaningless in terms of quantitation because some proteins can label far more efficiently than others, and some labeled proteins can lose affinity to their corresponding antibodies.

The sandwich assay format is an array analog of the widely applied ELISA technique. A number of variations of the sandwich assay technique exist, and all can be used in the practice of the disclosed methods. As used herein, “sandwich assay” is intended to encompass all variations on the basic two-site technique.

In a typical sandwich assay, an unlabeled capture antibody is immobilized onto a solid substrate and the sample to be tested brought into contact with the bound molecule. After a suitable period of incubation (i.e., a period of time sufficient to allow formation of an antibody-antigen complex), a second detection antibody labeled with a reporter molecule capable of producing a detectable signal is then added and incubated, allowing time sufficient for the formation of a ternary complex of antibody-labeled antibody. The detection antibody binds to the array only if the target protein is bound. Any unreacted material is washed way, and the presence of the antigen is determined by observation of a signal produced by the reporter molecule. The results can be qualitative, by simple observation of the visible signal, or can be quantitated by comparing with a control sample containing known amounts of cytokine and/or growth factor, for example.

Tissue cytokine levels must be detected in picogram quantities of short-lived species that reside in a very small and inaccessible interstitial volume. Thus, it is important to select an array method that yields the highest signal per unit sample volume. Sandwich arrays are particularly suited for the detection of proteins found in very low concentrations, such as cytokines and growth factors from biological specimens.

In some embodiments, unlabeled target cytokine and/or growth factor of interest in a biological sample is bound first by the immobilized capture antibody. A biotinylated detection antibody binds to the captured target protein, forming a capture-cytokine and/or growth factor target- detection antibody “sandwich”. The target is then detected indirectly by measuring the intensity of a streptavidin-conjugated label bound to the detection antibody.

“Detection antibody”, as used herein, is a protein comprising a detectable label which binds, is bound by, or forms a complex with one or more analytes of interest in a sample to be tested, or is a protein which binds, is bound by, or forms a complex with one or more analytes of interest which can be bound by further species that comprise a detectable label. Examples of detectable labels include chemically reactive labels, fluorescent labels, enzyme labels, and radioactive labels. In the present embodiments, detectable labels are fluorescent labels. A fluorescent dye that is compatible with commercial scanners and can be dried before scanning is generally used. In some embodiments, the label is streptavidin-Cy5 (SA-Cy5). In another particular embodiment, the label is streptavidin-Cy3 (SA-Cy3).

The detection antibodies used herein bind cytokines and/or growth factors. Anti-cytokine antibodies are representative non-limiting examples of both capture and detection proteins. Examples of suitable anti-cytokine antibodies include, but are not limited to, anti-human G-CSF, anti-human IL-10, anti-human GM-CSF, anti-human IL-13, anti-human GROα, anti-human IL-15, anti-human IFN-y, anti-human MCP-I, anti-human IL-1a, anti-human MCP-2, anti-human IL-2, biotinylated anti-human MCP-3, anti-human IL-3, biotinylated anti-human MIG, biotinylated anti-human IL-5, biotinylated anti-human/mouse/pig TGFβ1, anti-human IL-6, polyclonal rabbit anti-human RANTES, anti-human IL-7, biotinylated anti-human TNF-α, anti-human IL-8, anti-human TNF-β, monoclonal anti-human ENA-78 antibody, monoclonal anti-human 1-309 antibody, monoclonal anti-human IL-11 antibody, monoclonal anti-human IL-12 antibody, monoclonal anti-human IL-15 antibody, monoclonal anti-human IL-17 antibody, monoclonal anti-human M-CSF antibody, monoclonal anti-human MDC antibody, monoclonal anti-human MIP-Iα antibody, monoclonal anti-human MIP-1β antibody, monoclonal anti-human MIP-1 γ/Leukotactin antibody, monoclonal anti-human SCF antibody, monoclonal anti-human/mouse SDF-1 antibody, and monoclonal anti-human IL-4 antibody. In a further embodiment, the detection protein is an anti-IgA, anti-IgD, anti-IgE, anti-IgG, or anti-IgM antibody. In particular embodiments, detection antibodies are biotinylated according to methods that are known in the art.

Corresponding detection antibodies are prepared in a suitable diluent that does not interfere with binding between cytokines and/or growth factors and antibodies, and also prevents adhesion of detection antibodies to vessel surfaces. Conditions whereby an antigen/antibody complex can form are known in the art.

Common research equipment has been developed to perform high-throughput fluorescence detection, including instruments from GSI Lumonics (Watertown, Mass., United States of America), Amersham Pharmacia Biotech/Molecular Dynamics (Sunnyvale, Calif., United States of America), Applied Precision Inc. (Issauah, Wash., United States of America), Genomic Solutions Inc. (Ann Arbor, Mich., United States of America), Genetic MicroSystems Inc. (Woburn, Mass., United States of America), Axon (Foster City, Calif., United States of America), Hewlett Packard (Palo Alto, Calif., United States of America), and Virtek (Woburn, Mass., United States of America). Most of the commercial systems use some form of scanning technology with photomultiplier tube detection. Criteria for consideration when analyzing fluorescent samples are summarized by Alexay et al. (1996) The International Society of Optical Engineering 2705/63.

In order to mitigate any light scattering at the polymeric surface of a nitrocellulose slide while using a cDNA microarray scanner, the photo multiplier tube (PMT) gain can be optionally reduced. The thickness of the nitrocellulose surface can be taken into account by adjusting the focal length of the scanner.

In some embodiments provided herein are methods for detecting a binding that relies on absolute detection. For example, an initial diagnostic survey can comprise determining the presence or absence of binding of a cytokine to the microarray at a level as low as 1 ng/mL, as low as 100 pg/mL, as low as 5 pg/mL, or even as low as 1.0 pg/mL.

In some embodiments, data analysis also comprises characterization of assay performance features displayed by antibodies used in accordance with the disclosed methods.

Numerous software packages have been developed for microarray data analysis, and an appropriate program can be selected according to the array format and detection method. Some products, including ARRAYGAUGE™ software (Fujifilm Medical Systems Inc., Stamford, Conn., United States of America) and IMAGEMASTER ARRAY 2™ software (Amersham Pharmacia Biotech, Piscataway, N.J., United States of America), accept images from most microarray scanners and offer substantial flexibility for analyzing data generated by different instruments and array types. Other microarray analysis software products are designed specifically for use with particular array scanners or for particular array formats. A survey of representative non-limiting microarray analysis software packages can be found in Brush (2001) The Scientist 15:25-28. In addition, the guidance presented herein provides for the development of software and/or databases by one of ordinary skill in the art to facilitate analysis of data obtained by performing the method of the presently disclosed subject matter.

The foregoing detection methods can be used in methods for establishing a profile for evaluating wound status, such as but not limited for use in the diagnosis of wound status. In accordance with the present disclosure, a “wound” is any damage leading to a break in the continuity of the skin. Wound status is the condition of a wound, examined over a course of several hours to several days to several months, which provides an indication as to whether a wound is healing properly or is not.

In some embodiments, a profile of wound status is established by detecting in the wound the levels of cytokines and/or growth factors that are known to be associated with wound healing. In some embodiments, wound status is assessed by contacting wound fluid with a microarray for a time and under conditions sufficient to form an antigen-antibody complex. The binding is subsequently determined and the amount of complex formed is conventionally quantitated.

Wound status profiles can be established by measuring or detecting the levels of cytokine/growth factor in wound fluid. Further, wound status profiles can be compared to reference profiles by correlating the level of cytokine/growth factor found in a sample of wound fluid with standard or normal levels of plasma cytokine/growth factor. Such standards can be provided in a database as a reference wound status profile, and in some embodiments a wound status profile is compared to a reference profile to facilitate a diagnosis or to otherwise evaluate wound status.

In some embodiments, various consecutive wound samples are obtained so that cytokine and/or growth factor levels are conventionally quantitated over an appropriate period (for example, from about three days to about seven days, to about fourteen days, to about twenty-eight days, or even longer) to determine the changes in wound cytokine/growth factor levels over time. A wound status profile based on evaluation over time can also be established.

In a further embodiment, elevated wound cytokine/growth factor levels are compared to control or standard levels over a period of time. Such an embodiment can be directed to the diagnosis of a level of inflammation and/or infection at a wound site by quantitating the level of cytokine/growth factor present and comparing wound the cytokine/growth factor level with a standardized normal plasma cytokine/growth factor level. Such standards can be provided in a database as a reference wound status profile, and in some embodiments a wound status profile is compared to a reference profile to facilitate a diagnosis or to otherwise evaluate wound status.

According to the present methods, wounds can be diagnosed by repetitive immunoassay repeated over an effective period of time. An “effective period” of time is in some embodiments once a month for about two to about five months. An effective period is in some embodiments once a day for about three to about seven days. Once levels of cytokine/growth factor have been determined for a particular wound over an effective period of time, the practitioner can make an accurate assessment of the appropriate modalities to employ to optimize healing and minimize treatment costs. For example, wound cytokine/growth factor measurements taken over a several-day period will provide a baseline from which the practitioner can prescribe appropriate advanced care products to patients with indications of delayed or complicated wound healing. Advanced wound care products can also be employed prior to establishing a baseline if wound cytokine/growth factor levels are abnormal relative to standard levels.

Wound therapy assessment is greatly enhanced by the present methods and microarrays. In the past, the practitioner would treat a wound simply based on its outward appearance. With the presently disclosed subject matter, the practitioner can now quickly and more accurately determine the nature of a wound and prescribe an appropriate therapy for short-term remediation. The presently disclosed subject matter provides the practitioner with crucial information about the nature and extent of the wound. Aggressive wound therapy can now be implemented or avoided depending on the wound fluid cytokine/growth factor levels of a patient as determined by the presently disclosed subject matter.

As provided above, cytokines and growth factors are the principal mediators of wound healing and central to the fibrous capsule that forms around implanted materials. The time-dependent appearance of specific cytokines and growth factors and their respective levels can be correlated with specific wound healing stages based on histology of the surrounding tissue.

Also disclosed herein are kits for establishing a profile of one of host organism response to foreign, implanted material, wound healing, and both host organism response to foreign, implanted material and wound healing. In some embodiments the kits comprise:
at least one microarray for the detection of cytokines and growth factors associated with one of host organism response to foreign, implanted material, wound healing, and both host organism response to foreign, implanted material and wound healing, wherein the microarray comprises a plurality of capture antibody samples immobilized on a solid substrate to form a plurality of array elements, wherein:
(i) each capture antibody sample comprises an anti-cytokine or an anti-growth factor capture antibody in a printing buffer solution; and
(ii) each anti-cytokine or an anti-growth factor capture antibody specifically binds a cytokine or growth factor associated with one of host organism response to foreign, implanted material, wound healing, and both host organism response to foreign, implanted material and wound healing;
at least one reagent useful for the detection of cytokines and growth factors associated with one of host organism response to foreign, implanted material, wound healing, and both host organism response to foreign, implanted material and wound healing; and
instructions for establishing a profile of one of host organism response to foreign, implanted material, wound healing, and both host organism response to foreign, implanted material and wound healing based on the binding.

The instructions can be written instructions, computer readable instructions (which can be embodied in a computer readable medium), and/or both written materials and computer readable instructions.

Thus, the present disclosure provides kits that are useful for establishing profiles for evaluating wound healing, as well as kits that are useful for establishing profiles for determining the biocompatibility of implantable materials. The term “kit” refers to assemblies of diagnostic apparatus and components for performing the cytokine/antibody detection methods disclosed herein.

In some embodiments, a kit comprises a microarray specific for the detection of cytokines and/or growth factors associated with wound healing. In some embodiments, a kit comprises a microarray specific for the detection of cytokines and/or growth factors associated with biocompatibility of implantable materials. Microarrays included in the kits are produced according to the printing techniques set forth herein. The kit can comprise an optimized blocking buffer, as set forth herein. The kit can comprise detection antibodies, which can be labeled or unlabeled.

The kit can optionally be compartmentalized, and can be constructed to include a first structure containing the microarray of capture anti-cytokine and/or anti-growth factor antibodies as defined herein, and one or more additional containers comprising blocking buffers, detection antibodies, and/or fluorescent labels, respectively. The detection antibody can be labeled with a reporter molecule or the detection antibody can be unlabeled. The unlabeled antibody can be conventionally modified by the kit user to include a reporter molecule (i.e., label).

The kit can optionally also include, for example, a container of cytokine/growth factor as a solution at a known concentration to act as a standard or positive control. Additional containers can optionally contain substrates or reagents appropriate for visualization of the fluorescent label.

The kit can also include written materials, computer readable instructions (which can be embodied in a computer readable medium), and/or both written materials and computer readable instructions such as notification of approved uses and instructions therefor, including specific information correlating elements of the microarray with target cytokines or growth factors. Moreover, the kit can contain specific indicators identifying the significance of the presence or absence of a binding event when a biological sample is exposed to the microarray and a measurable signal or detection of the antibody-antigen binding event is present. The kit can also include an index or key pursuant to which each element of the array can be correlated to a target cytokine or is part of a database that is correlated to the specific members of the array, or a collection of members of the array. In particular embodiments, the specific array elements and their corresponding target cytokines and/or growth factors are correlated to a wound status assessment or an evaluation of biocompatibility.

### EXAMPLES

The following Examples provide illustrative embodiments. Certain aspects of the following Examples are disclosed in terms of techniques and procedures found or contemplated by the present inventors to work well in the practice of the embodiments. In light of the present disclosure and the general level of skill in the art, those of skill will appreciate that the following Examples are intended to be exemplary only and that numerous changes, modifications, and alterations can be employed without departing from the scope of the presently claimed subject matter.

### Example 1

### Cytokines, Growth Factors, and Antibodies

Capture antibodies, cytokines, growth factors, and detection antibodies were all purchased from R& D Systems, Minneapolis, Minn., United States of America. The capture antibodies used were monoclonal anti-human IL-1β antibody, monoclonal anti-human TNF-α antibody, anti-human VEGF antibody, monoclonal anti-human MIP-1β antibody, and monoclonal anti-TGF-β1, -β2, β3 antibody. The cytokines and growth factors used were recombinant human IL-1β, recombinant human TNF-α, recombinant human VEGF, recombinant human MIP-1β, and recombinant human TGF-β1. The detection antibodies used were biotinylated anti-human IL-1β antibody, biotinylated anti-human TNF-α antibody, biotinylated anti-human VEGF antibody, biotinylated anti-human MIP-1β antibody, biotinylated anti-human TGF-β1 antibody.

30% glycerol and 5 mM EDTA were obtained from Sigma (St. Louis, Mo., United States of America). 70% PBS was obtained from Invitrogen Corp. (Carlsbad, Calif., United States of America).

### Example 2

### General Assay Protocol

Capture antibodies were dissolved in one of three array printing buffers (see Example 4 below) at a concentration of 250 μg/ml, and then transferred into a 96-well plate before printing. A MICROSYS™ 5100 microarrayer (Cartesian Technologies, Irvine, Calif., United States of America) was used to print anti-human cytokine capture antibodies and control spots on microscope slides. Printing was carried out in an atmospherically isolated chamber with a relative humidity of 70% at room temperature. CHIPMAKER™ microarray pins, model CMP4 (TeleChem, Sunnyvale, Calif., United States of America), were used for arraying. The pin delivered 1.0 nL antibody solution per printed spot, and the diameter of a spot was 160 μm. Given a molecular weight of 155 kilodaltons, a rough estimation of the antibody density was 5×10¹² molecules/cm². Prior to printing, pins were cleaned in absolute alcohol in an ultrasonic bath for 5 minutes and dried in a stream of N₂.

Six arrays of eight rows by ten columns were generated on various putative slide surfaces with a pitch of 500 μm to form an array. In each array, row 1 was biotin labeled bovine serum albumin (biotin-BSA; Sigma) and regarded as the “detection control” (also called a “positive control” or “orientation row”); row 2 was bovine serum albumin (BSA; Invitrogen), regarded as the “negative control”; rows 3-7 were respectively capture antibodies for human IL-1β, TNF-α, VEGF, MIP-1β and TGF-β1; row 8 was biotin-SP-conjugated AffiniPure F(ab′)₂ fragment goat anti-human IgG (biotin-GAH IgG; Jackson ImmunoResearch Laboratories Inc., West Grove, Pa., United States of America) used as another detection control. All the arrays used this pattern unless indicated otherwise. After printing, all the slides were kept in a humid chamber at room temperature for post-print incubation before further treatment: 3-hour incubation for glass slides and 1.5-hour incubation for nitrocellulose slides. A corral was drawn around each array using a hydrophobic SUPER PAP PEN HTTM (Research Products International Corp., Mt. Prospect, Ill., United States of America) to contain the incubation and washing solutions.

The general assay procedure used in Examples 3 to 9 was as follows:

1. Array-containing slides were washed with wash buffer (PBS with 0.05% Tween 20 (Calbiochem, San Diego, Calif., United States of America), aspirated, and then blocked one of three blocking buffers (see Example 5, below) for 1.5 hours.

2. After aspiration of the blocking buffer, 50 μl of 10 ng/ml cytokine cocktail (a combination of the above-mentioned five cytokines) prepared in a diluent (1.4% delipidized bovine serum (R&D System), 0.05% Tween 20 in Tris-buffered saline) was added onto each array and incubated in a humidity chamber at room temperature for 1 hour.

3. After repeating the aspiration/wash step, 50 μl of detection antibody cocktail (a combination of the above-mentioned 5 cytokine detection antibodies at 1:500 dilution) was added onto each array and incubated for another hour.

4. After repeating the aspiration/wash step, 50 μl of streptavidin-Cy5 (SA-Cy5; CalTag Laboratories, Burlingame, Calif., United States of America) at a 1:50 dilution was added to each array and incubated for 30 minutes in the dark, washed again and dried in a stream of N₂.

5. Dried slides were immediately scanned and imaged using a GENEPIX® 4000B microarray scanner and GENEPIX® Pro software (Axon Instruments, Union City, Calif., United States of America). Data were also acquired and analyzed using the same software. In analysis, all the fluorescent intensities were background corrected mean fluorescence intensities of the pixels within the spot ellipse. For spots (features), the median value did not accurately reflect spot (feature) intensity because the images were non-uniform. Therefore, the mean intensity was used for the spots.

The above procedure, henceforth referred to as the “general protocol”, was used to select the solid support, array printing buffer, blocking buffer, and fluorescent dye most optimal for the detection of cytokines and growth factors. Specific modifications to the general protocol are annotated in the following examples.

### Example 3

### Selection of Optimal Solid Substrate for Cytokine/Growth Factor Microarray Detection

Capture antibodies were covalently bound to the following solid substrates (“slides”): SuperEpoxy slides (TeleChem, Sunnyvale, Calif., United States of America), Silylated slides (also called Aldehyde slides, Cel Associates, Pearland, Tex., United States of America), Poly-L-lysine slides (Cel Associates), and silanated slides (also called Amine slides, Cel-Associates). Capture antibodies were non-covalently bound to FAST® slides (glass slides coated with a proprietary nitrocellulose microporous polymer, also called nitrocellulose slides, Schleicher & Schuell BioScience, Keene, N.H., United States of America). The water contact angles for each slide surface were measured by a goniometer (Rame-hart, Mountain Lakes, N.J., United States of America). The general protocol for array preparation and assay was applied to each slide as set forth above, and the quality of array images was used to select the best slide for the cytokine detection arrays.

The quality of capture antibody spotted onto commercially available slides was compared and the results are set forth in Table 1. All the glass slides had different levels of smearing and spreading. Nitrocellulose slides are microporous substrates that readily hold the spotted antibody and show no evidence of smearing or spreading. A common concern with the covalently bound proteins is a loss of reactivity due to orientation or chemical modification of the active site. Based on the foregoing, it was concluded that that non-covalent protein binding slides, such as microporous nitrocellulose, performed better in cytokine detection arrays than covalent protein binding glass slides. The three-dimensional surface of the slides absorbs and holds spots of capture antibody of higher than 150 μg/ml, which has been disclosed as the upper limit for glass slides. See B. A. Stillman et al., BioTechniques (2000) 29, 392.

TABLE 1
Quality of Capture Antibody Spots Arrayed Various Solid Substrates

| | Water | | |
|---|---|---|---|
| | contact | Printing | |
| Substrate | angle (deg) | Results | Comments |
| FAST ® slides | 70 ± 2* | Drop held | Microporous surface |
| (nitrocellulose | | rapidly | prevents smearing and |
| microporous) | | upon | spreading of spots by |
| | | deposition | absorption and non- |
| | | | covalent binding; |
| | | | antibody can be loaded |
| | | | in high amounts. |
| SuperEpoxy slides | 43 ± 2 | Smearing | Epoxide group very |
| (Epoxy) | | and | reactive, proteins |
| | | spreading | bound very tightly to |
| | | | surface, significant |
| | | | smearing and |
| | | | spreading of spots, |
| | | | highest sensitivity of |
| | | | glass slides tested. |
| Silylated slides | 40 ± 1 | Smearing | Aldehyde group very |
| (Aldehyde) | | and | reactive, rinsing and |
| | | spreading | blocking cause |
| | | | smeared antibodies to |
| | | | bind to surface, |
| | | | especially when the |
| | | | capture antibody |
| | | | concentration is high, |
| | | | moderate sensitivity. |
| Poly-L-lysine | 41 ± 2 | Relatively | Polar amine is less |
| slides | | low signal; | reactive than aldehyde, |
| | | Smearing | Smearing around the |
| | | and | spots is reduced, but |
| | | spreading | less antibody is bound |
| | | | to surface, moderate to |
| | | | low sensitivity. |
| Silanated slides | 31 ± 1 | Relatively | Polar amine reactive |
| (Amine) | | low signal; | group, similar |
| | | Some | smearing and |
| | | smearing | moderate to low |
| | | and | sensitivity to Poly-L- |
| | | spreading | lysine slides. |

### Example 4

### Selection of Optimal Printing Buffer for Cytokine/Growth Factor Microarray Detection

Unlike cDNA or oligonuclotides, capture antibodies can denature during array printing, so the selection of appropriate printing and blocking buffers is critical. Standard PBS array printing buffers quickly evaporate, even in room temperature and high humidity environments, and can cause denaturation of capture antibodies printed on slides or in microtiter plates. Glycerol is often used to increase solubility of amphiphilic proteins in array printing buffer and to reduce evaporation after printing. G. MacBeath and S. L. Schreiber, Science (2000) 289, 1760. However, glycerol can cause other problems, particularly on glass slides. For example, highly viscous glycerol can retard precipitation of capture antibodies to the solid substrate surface, which can permit the subsequent rinse and blocking steps to cause the unevaporated antibody droplet to bind as a smeared spot to solid substrate surfaces. The effect of smearing can be pronounced at high capture antibody concentrations.

The general assay protocol set forth above was used to determine the optimal printing buffer for the selected cytokines and growth factors. The capture antibody solution was prepared using one of the three following printing buffers: (1) in-house array printing buffer (30% glycerol/70% PBS/5 mM EDTA); (2) 1× PBS; or (3) a commercially available array printing buffer recommended by Schleicher & Schuell. The final images associated with different array printing buffers were used to select the best buffer for the cytokine detection arrays.

Table 2 shows the effects of array printing buffers on quality of printed capture antibody spots. 70% PBS/30% glycerol/EDTA was selected as array printing buffer for the cytokine detection protein arrays because it produced the most uniform spots, and prevented droplet evaporation and capture antibody denaturation.

TABLE 2
Effects of Array Printing Buffer on Quality of Printed Antibody Spots

| | Surfaces | | |
|---|---|---|---|
| Buffer | examined | Results | Comments |
| PBS | FAST ®, | Small, irregular | Droplet |
| | SuperEpoxy, | and contracted | contraction |
| | Silylated, Poly- | spots with low | caused by rapid |
| | L-lysine, | fluorescent | evaporation. |
| | Silanated | intensity | Small droplet size |
| | slides | | yields low |
| | | | fluorescent |
| | | | counts. |
| Schleicher & | FAST ® slide | Uniform spots, | Similar to PBS |
| Schuell array | only | Consistent | problems with |
| buffer* | | fluorescent | drop evaporation, |
| | | intensity | must be printed in |
| | | | high humidity |
| | | | environments. |
| 70% PBS/30% | FAST ®, | Uniform spots, | Glycerol prevents |
| glycerol/EDTA | SuperEpoxy, | Consistent | rapid drop |
| | Silylated, Poly- | fluorescent | evaporation of |
| | L-lysine, | intensity | solutions on slide, |
| | Silanated | | in sample well and |
| | slides | | in transit. |

### Example 5

### Selection of Optimal Blocking Buffer for Cytokine/Growth Factor Microarray Detection

PBS/BSA and PBS/nonfat milk are commonly used to block non-specific binding, but are not suitable for nitrocellulose slides. Improper blocking agents can contribute to fluorescent background, so selection of an optimal blocking buffer for use with the present arrays was carried out.

The general protocol was used. Three blocking buffers were used to block arrays on different substrates: PBS/1% BSA, a commercially available blocking buffer recommended by Schleicher & Schuell, and an in-house blocking buffer (PBS containing 3% Tween 20, 5% sucrose and 0.1% NaN₃)

Table 3 shows the effects of three array blocking buffers on capture antibody arrays. The in-house blocking buffer was selected as the blocking buffer for cytokine detection arrays, because of minimum background fluorescence. Although not wishing to be bound to any particular theory, sucrose and Tween 20, a nonionic surfactant, appear to effectively stick to the nitrocellulose surface and block unoccupied protein binding sites. M. Steinitz, Analytical Biochemistry (2000) 282, 232; E. Harlow and D. Lane (1988) Antibodies: a Laboratory Manual, First Edition (Cold Spring Harbor Laboratory Press, Cold Spring Harbor, N.Y., United States of America), Chapter 12.

TABLE 3
Effects of Array Blocking Buffer on Antibody Arrays

| | Surfaces | | |
|---|---|---|---|
| Blocking buffer | examined | Results | Comments |
| PBS/1% BSA* | FAST ®, | High | BSA adsorbed to |
| | SuperEpoxy, | background | nonarrayed space |
| | Silylated, Poly- | fluorescence | on FAST ® slides |
| | L-lysine, | found on | was intrinsic |
| | Silanated | FAST ® | fluorescence |
| | slides | slides | |
| Schleicher & | FAST ® slides | Moderate | Improvement over |
| Schuell blocking | only | background | BSA blocking, but |
| buffer** | | fluorescence | still significant |
| | | | background. No |
| | | | appreciable benefit |
| | | | to signal intensities |
| | | | at low antibody |
| | | | concentrations |
| 5% Sucrose/ | FAST ®, | Little to no | Electrostatic |
| 3% Tween 20 | SuperEpoxy, | background | interaction of the |
| | Silylated, Poly- | fluorescence | sucrose, Tween 20 |
| | L-lysine, | | and slide substrate |
| | Silanated | | |
| | slides | | |

### Example 6

### Selection of Optimal Fluorescent Dye for Cytokine/Growth Factor Microarray Detection

The general protocol was applied, using one of the following fluorescent dyes: SA-Cy5, Streptavidin-Cy3 (SA-Cy3; Caltag Laboratories, Burlingame, Calif., United States of America), Streptavidin-Phycoerythrin-Cyanine 5 (SA-PC5; Immunotech), Streptavidin-Phycoerythrin (SA-PE; CalTag Laboratories), Streptavidin-Fluorescein Isothiocyanate (SA-FITC; CalTag Laboratories). Fluorescent intensity in scanned images and compatibility of dyes with cDNA microarray scanner were used to select the optimal fluorescent dye.

Table 4 shows the comparison of different fluorescent dyes tested. SA-Cy5 could be detected by a conventional cDNA microarray scanner, and was sensitive to cytokine detection with limit down to 10 pg/ml, making it the most suitable dye selection.

TABLE 4
Selection of Different Fluorescent Dyes

| | | λ excitation, λ | Compatible with |
|---|---|---|---|
| | Image | emission | green/red DNA |
| Dye | quality | (nm) | scanner |
| Streptavidin- | Excellent, | 649, 670 | Yes |
| Cyanine 5 (SA- | down to | | |
| Cy5) | 10 pg/ml | | |
| Streptavidin- | Good, | 550, 570 | Yes |
| Cyanine 3 (SA- | down to | | |
| Cy3) | 100 pg/ml | | |
| Streptavidin- | Good, | 486-580, 675 | Yes |
| Phycoerythrin- | down to | | |
| Cyanine 5 (SA- | 100 pg/ml | | |
| PC5) | | | |
| Streptavidin- | Poor, | 566, 575 | No |
| Phycoerythrin (SA- | weak | | |
| PE) | signal | | |
| Streptavidin- | Poor, | 494, 525 | No |
| Fluorescein | weak | | |
| Isothiocyanate (SA- | signal | | |
| FITC) | | | |

### Example 7

### Selection of Optimal Cytokine Capture Antibody Concentration for Cytokine/Growth Factor Microarray Detection

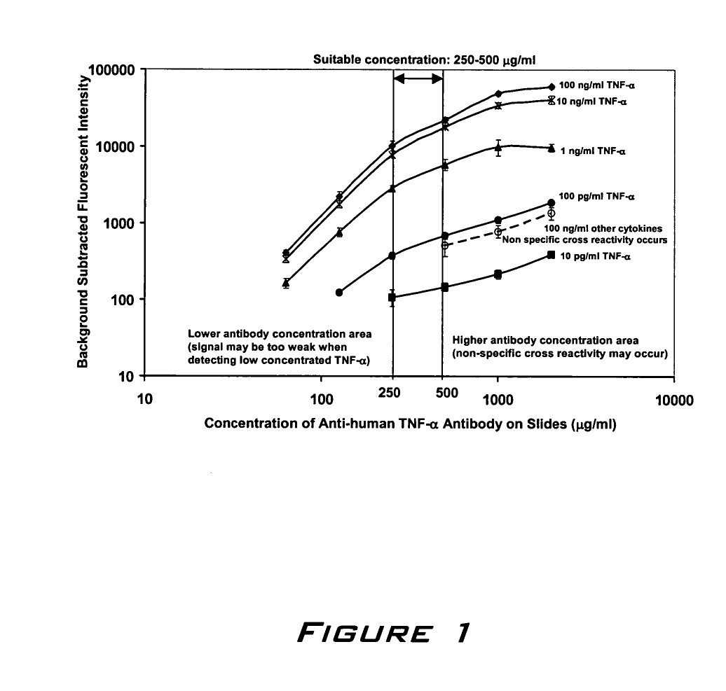

Optimal capture antibody concentration was determined by individual design rather than simply adapting cDNA microarray protocol. Generally, the higher the cytokine capture antibody concentration, the higher the sensitivity of the cytokine detection arrays. However, high concentrations of capture antibody also lead to non-specific cross reactivity. Merely maximizing the capture antibody concentration can lead to problems with the saturation of fluorescent intensities, as shown in FIG. 1. Moreover, even when there is no risk of non-specific cross reactivity (e.g., in a standard singleplex cytokine detection system), the capture antibody concentration should not be too high because an antibody-antigen affinity bonding is a dynamic equilibrium, and highly concentrated capture antibodies can alter significantly target proteins concentration in a sample. M. F. Templin et al., Trends in Biotechnology (2002) 20,160.

According to the results of previous comparisons, FAST® nitrocellulose solid substrate, an array printing buffer of 70% PBS/30% glycerol/EDTA, a blocking buffer of 5% Sucrose/3% Tween 20, and the fluorescent dye SA-Cy5 dye were most suitable to our cytokine detection arrays. These conditions were used to optimize the cytokine capture antibody concentration for printing the arrays. TNF-α was selected as an example for identifying capture antibody concentrations, as follows.

Monoclonal anti-human TNF-α antibody was dissolved in array printing buffer at concentration of 62.5 μg/ml, 125 μg/ml, 250 μg/ml, 500 μg/ml, 1 mg/ml, and 2 mg/ml, respectively. The array pattern here was different from the pattern in general protocol. In each array, row 1 represented biotin-BSA detection control, and row 2 was BSA negative control; rows 3-8 respectively represented the corresponding 62.5 μg/ml to 2 mg/ml capture antibodies. In order to reduce the variations caused by different array-containing slides, six identical arrays were printed on one single FAST® slide, and separated in corrals drawn by a hydrophobic pen.

Array 1 was incubated for 1 hour with 50 μl of 100 ng/ml cytokine cocktail only without TNF-α to establish the relationship between non-specific cross reactivity and capture antibody concentration. All other arrays (2-6) were respectively incubated for 1 hour with 50 μl of 100 ng/ml, 10 ng/ml, 1 ng/ml, 100 pg/ml, and 10 pg/ml TNF-α in order to detect the relationships between target TNF-α concentrations, anti-TNF-α capture antibody concentrations, and background subtracted fluorescent intensities of the spots on the scanned images. The remaining steps followed the general protocol. Each data point of the resulting curves in this test, as well as in the following tests, was obtained from averaging the intensities of at least eight spots (features).

The solid curves in FIG. 1 show the relationships between the concentrations of anti-TNF-α capture antibody, the concentrations of target TNF-α samples, and the background subtracted fluorescent intensities. Generally, background subtracted fluorescent intensities for all the capture antibody spots increased with increasing capture antibody concentration. However, the curves saturate at high capture antibody concentration, such as 1-2 mg/ml. When detecting highly concentrated target TNF-α, such as 100 ng/ml, even capture antibody spots produced from low concentration solutions can have significant fluorescent intensity. When detecting very low concentrated target TNF-α, such as 10 pg/ml, no significant fluorescent intensity was observed from the spots produced from the same concentrated antibody solutions. Only spots made by 250 μg/ml of capture antibody or higher consistently had significant fluorescent intensity

The experiment for which the data presented in FIG. 1 for anti-TNF-α was repeated for anti-IL-1β, anti-VEGF, anti-MIP-1β, and anti-TGF-β1, yielding virtually the same results. In view of all results, the optimal capture antibody concentration for the cytokine/growth-factor detection arrays lies between about 250 and about 500 μg/ml. This range of capture antibody concentration allows detection of very dilute target protein samples, but also avoids non-specific cross reactivity.

### Example 8

### Non-Specific Cross Reactivity Test of Optimized Microarray

Based on the results of all the previous experiments on slides, buffers, fluorescent dyes, and identification of capture antibody concentration, 250 μg/ml anti-cytokine capture antibodies were arrayed on FAST® slides to build the optimized cytokine detection arrays. Each FAST® slide contained six separated identical arrays. The pattern and size of each array was already disclosed in the general protocol. Arrays 1-5 were incubated for 1 hour with 100 ng/ml of human IL-1β, TNF-α, VEGF, MIP-1β, and TGF-β1, while Array 6 was incubated with a 100 ng/ml cocktail of all five cytokines. The remaining steps followed the general protocol.

The dashed curve in FIG. 1 contains the response of the anti-TNF-α spots to a 100 ng/ml cocktail of multiple cytokines, deliberately omitting TNF-α. When anti-TNF-α concentration reached or exceeded 500 μg/ml, non-specific cross reactivity occurred.

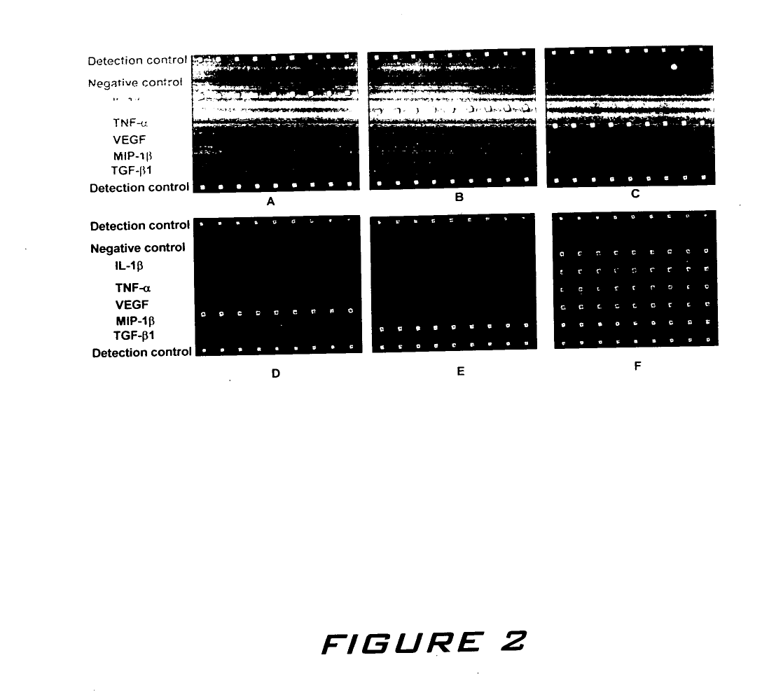

FIG. 2 shows the non-specific cross reactivity test on the optimized cytokine detection array. From Arrays 1-5, all binding occurred only at the specific capture antibody sites (FIG. 2, A-E). In Array 6 (FIG. 2, F), all five kinds of capture antibody spots simultaneously bound their specific target cytokines. The results suggested the optimized cytokine detection arrays do not have non-specific cross reactivity.

### Example 9

### Generation of Standard Curves With Optimized Cytokine Detecting Arrays

Arrays 1-5 were incubated with 100 ng/ml, 10 ng/ml, 1 ng/ml, 100 pg/ml, and 10 pg/ml cytokine cocktail respectively, while Array 6 was incubated with diluent (1.4% delipidized bovine serum, 0.05% Tween 20 in Tris-buffered saline), and regarded as a negative control. The remaining experiment was carried out according to the general protocol. The resultant image maps were used to build sigmoid plots for all the five cytokines, and relevant portions of the standard curves were identified.

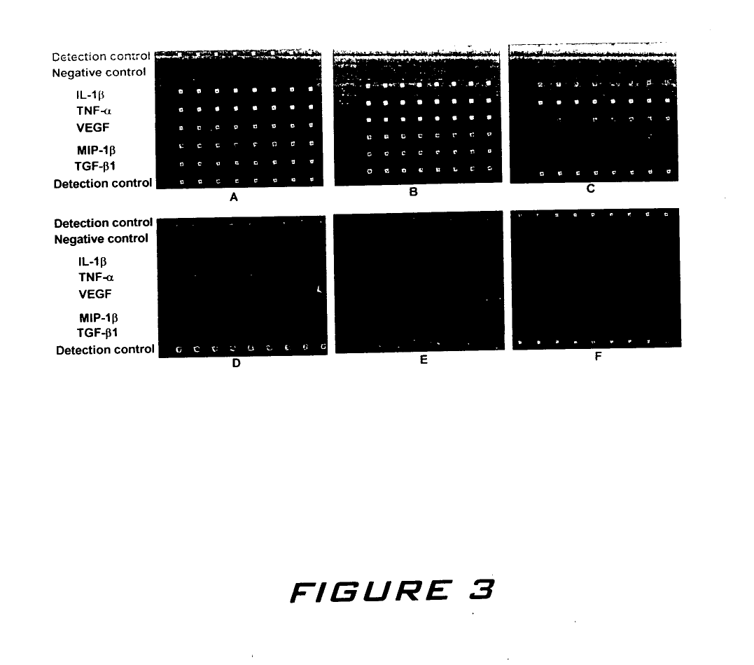

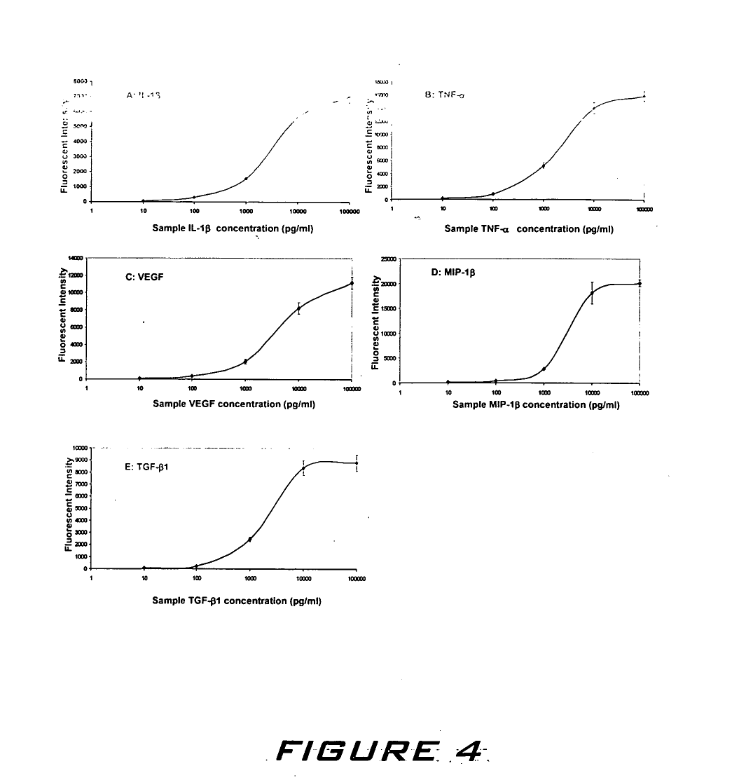

FIG. 3 shows the results of a cocktail of all five cytokines in a dose-response format on the cytokine detection arrays. As concentration of the cytokine cocktail decreased, the fluorescent intensity for all cytokine capture antibody spots decreased. The corresponding sigmoid curves for each cytokine and growth factor are shown in FIG. 4. The standard curves are relevant between the concentrations of 10 pg/ml and 10 ng/ml. The fitting parameters of the linear regions for the five cytokines are listing in Table 5. All five cytokines yielded standard curves suitable for quantitating the levels of the cytokines from an experimental sample.

TABLE 5
Fit Data for the Regression of log (Fluorescent Intensity) with log
(concentration) for the Five Cytokines in the Assays. Log F = K log C + B

| | Linear range | | | |
|---|---|---|---|---|
| Cytokine | (pg/ml) | K | B | R² |
| IL-1β | 10-10000 | 0.6823 | 1.076 | 0.995 |
| TNF-α | 10-10000 | 0.6739 | 1.551 | 0.985 |
| VEGF | 10-10000 | 0.6889 | 1.189 | 0.998 |
| MIP-1β | 10-10000 | 0.8107 | 1.007 | 0.999 |
| TGF-β1 | 10-10000 | 0.7816 | 0.8567 | 0.974 |

### Example 10

### In Vitro VEGF Release Study and Cytokine Expression Levels in Human Sera

The optimized cytokine detection array was incubated directly with 50 μl PBS solution into which VEGF was released from a HEMA-based hydrogel intended to induce angiogenesis in the wound healing bed surrounding implanted sensors. The assay was carried out according to the general protocol.

Human patient sera were obtained anonymously from the Clinical Immunology Lab and the Oncology Division, Duke University Medical Center. After being taken out of a −80° C. freezer and thawed, two patients' sera were directly incubated with the optimized cytokine detection arrays. The tests were carried out according to the general protocol.

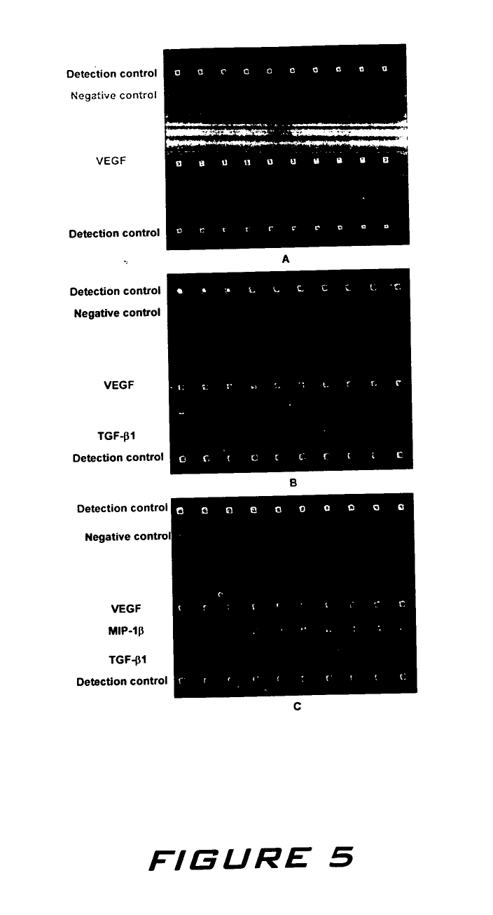

FIG. 5 demonstrates the ability of the cytokine detection array to quantitatively detect cytokine concentrations in various samples. Array A shows the response to VEGF released in vitro from a hydrogel into buffer solution. VEGF in that sample was 9.08±0.35 ng/ml. Arrays B and C were exposed to human sera from two patients' samples. Patient #1 had 133±36 pg/ml VEGF, less than 10 pg/ml TGF-β1, and negligible amounts of the other 3 cytokines. Patient #2 had 600+100 pg/ml VEGF and 15±5 pg/ml MIP-1β. TGF-β1 was detected, but less than 10 pg/ml. Patient #2 had undetectable levels of IL-1β and TNF-α.

### Examples 11-15

### Overview: Direct Comparison of Direct Labeling and Sandwich Immunoassays on the Same Optimized Microarray

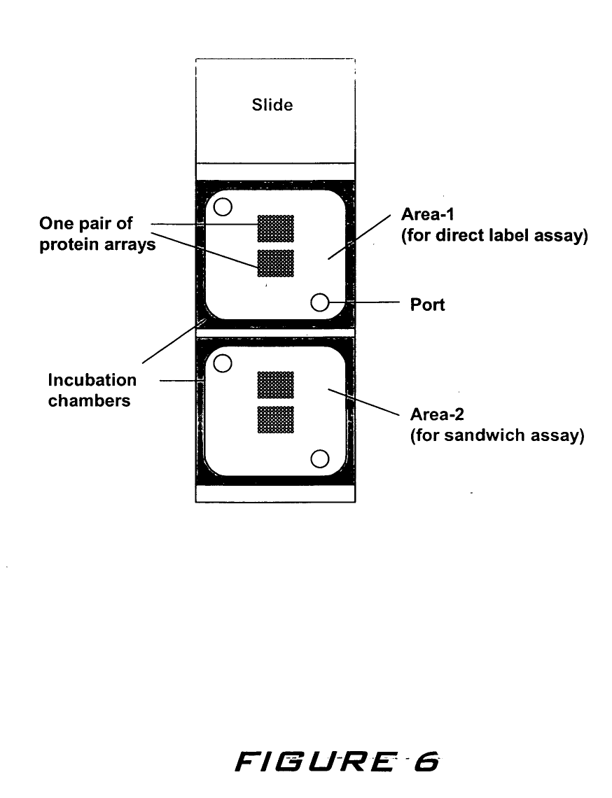

Direct labeling and sandwich protein assays were carried out in parallel for five model cytokines (IL-1β, TNF-α, VEGF, MIP-1β, and TGF-β1) on four different array printing slides. In order to minimize slide-to-slide variability, which can pose a major problem in the development of microarray-based technology, each slide was separated into two measurement regions with disposable incubation chambers that allowed direct label and sandwich assays to be performed in parallel on the same slide (FIG. 6). This ensured that each format comparison was conducted under the same experimental conditions of array printing, washing, and scanning.

### Example 11

### Materials

All monoclonal capture antibodies, cytokines, growth factors, and biotinylated detection antibodies were obtained from R&D Systems (Minneapolis, Minn., United States of America). Solid substrates used were FAST® slides (glass slides coated with microporous nitrocellulose, Schleicher & Schuell BioScience, Keene, N.H., United States of America), SuperEpoxy slides (TeleChem, Sunnyvale, Calif., United States of America), silylated slides (Cel Associate, Pearland, Tex., United States of America), and ALS Aldehyde slides (Cel Associates). Secure-Seal SA200 incubation chambers were obtained from Schleicher & Schuell (Keene, N.H., United States of America). Buffers, including array-printing buffers, blocking buffers, and wash buffers, were prepared as disclosed above.

### Example 12

### Microarray Fabrication

As shown in FIG. 6, each slide was printed with two pairs of four identical 8×10 arrays. Each pair of arrays was isolated by incubation chambers into two separate incubation areas. Direct label assays were performed in area 1. Sandwich assays were performed in area 2. Rows 1 and 8 were biotinylated bovine serum albumin, and were regarded as the detection rows. Row 2 was bovine serum albumin, which was regarded as the negative control. Rows 3-7 were, respectively, capture antibodies for human IL-1β, TNF-α, VEGF, MIP-1β, and TGF-β1. A Microsys 5100 microarrayer (Cartesian Technologies, Irvine, Calif., United States of America) was used to print anti-human cytokine capture antibodies and control spots on microscope slides. Printing was carried out in an atmospherically controlled chamber with a relative humidity of 70% at room temperature. Chipmaker microarray pins, model CMP4 (TeleChem, Sunnyvale, Calif., United States of America), were used for arraying spots of 160 μm-diameter. Prior to printing, the pin was cleaned in absolute alcohol in an ultrasonic bath for 5 minutes and dried in a stream of nitrogen.

### Example 13

### Cytokine Direct Labeling Using Gel Filtration of Free Dye

Gel filtration column chromatography was used to separate free dye in a direct labeling protein detection assay. A protocol suitable for labeling low concentration cytokines and growth factors was developed based on the specification of the Fluorolink MAb Cy3 labeling kit (Amersham Biosciences, Piscataway, N.J., United States of America). One foil packet from the labeling kit containing dried Cy3 dye was dissolved in 10 μL of DMSO. The dissolved dye was aliquoted to ten 1 μL tubes. 100 ng/mL cytokine solution was combined with a 40 μg/mL solution of bovine serum albumin (BSA), where the BSA was used as carrier protein to prevent cytokine over labeling. 5 μL of coupling buffer were added to 100 μL of cytokine solution and mixed thoroughly by gentle vortexing or by repeated pipetting. The entire volume of protein and coupling buffer was transferred to 1 μL reactive dye-containing tubes, capped, and mixed thoroughly. The mixture was incubated at room temperature for 30 minutes mixing approximately every 10 minutes. The mixture of labeled cytokine and unreactive dye was poured over Dextran gel filtration columns, where the first band passing through the column contained the labeled cytokine. This labeling protocol was optimized by comparing the final signal intensities on protein arrays, checking desalted protein sample concentration with Pierce's BCA protein assay kit (Pierce Biotechnology, Inc., Rockford, Ill., United States of America), and estimating dye to protein molar ratios.

### Example 14

### Direct and Sandwich Array Measurements

To reduce slide-to-slide variation, direct label and sandwich format assays were performed on separate areas of the same slide designated as area-1 and area-2 (FIG. 6). Both sandwich and direct label array measurements began with the same cytokine concentrations of 100 ng/mL. The sandwich assays used the cytokine cocktail sample directly without modification. For direct label assays, the 100 ng/mL of original cytokine sample was fluorescently labeled and then used as collected from the gel filtration column.

After sealing the two areas with incubation chambers, area-1 and area-2 were incubated with 250 μL blocking buffer for at least 2 hours. Keeping the blocking buffer in area-1, the blocking buffer in area-2 was removed via a aspiration port on the chamber, and 250 μL of the target cytokine cocktail for sandwich assay (100 ng/mL) was added into area-2. After 1 hour of incubation, the used incubation chamber was removed from area-2, the target cytokine cocktail was aspirated, and area-2 was washed with wash buffer. Aspiration/wash was repeated 3 times. A new incubation chamber was placed on area-2, and incubated for 1 hour with a cocktail containing the five detection antibodies. The detection antibody cocktail was diluted 1:250 from stock concentrations (100 μg/mL). Aspiration/wash was repeated 3 times, followed by placement of a new incubation chamber and incubation of area-2 with 250 μL of streptavidin-Cy3. At this time, the blocking buffer in area-1 was aspirated from the chamber covering area-1, and 250 μL of the target cytokine cocktail for direct label was added and incubated with area-1 for 30 minutes in the dark. Finally, both chambers were removed from area-1 and area-2, and each area was aspirated, washed, and dried in a stream of nitrogen. Dried slides were immediately scanned and imaged using a GENEPIX® 4000B microarray scanner and GENEPIX® Pro software (Axon Instruments, Union City, Calif., United States of America). All the fluorescent intensities in scanned images were displayed as background corrected mean fluorescence intensities of the pixels within the spot ellipse. Median background values were used for background, as sampling was assumed to be uniformly distributed throughout the background. For spots, the median value did not accurately reflect spot intensity because the images were non-uniform. Therefore, the mean intensity was used for the spots.

### Example 15

### Same-Slide Comparison of Direct and Sandwich Assays

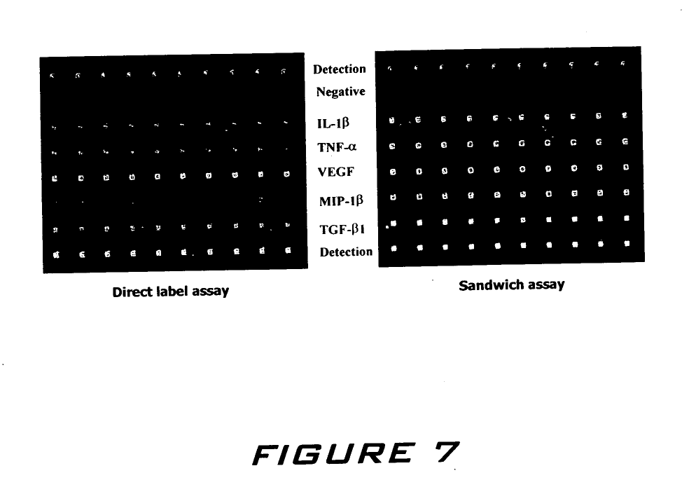

Examples of direct and sandwich arrays scanned on the same slide are shown in FIG. 7. Spot intensities from the sandwich assays were visually more distinctly above background than were the corresponding spots of the direct label assay. In this example, only the signal from VEGF appears to be of similar intensity for the two formats.

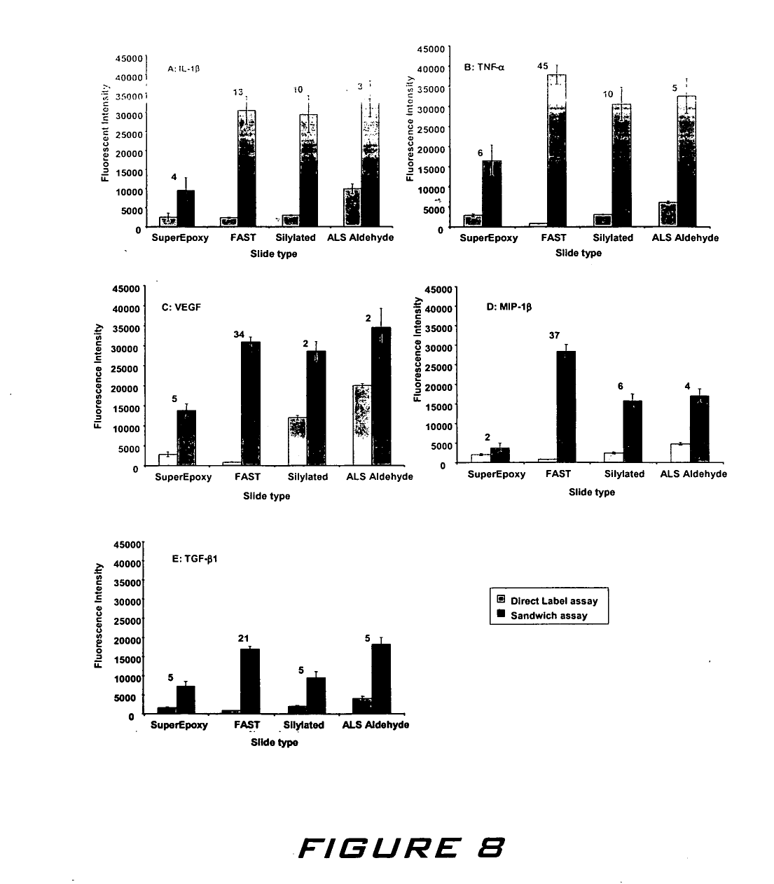

FIG. 8 summarizes the background subtracted cytokine signals for the two format assays on the four different slides examined. Numbers at the top of the bars are the ratio of the background subtracted fluorescent intensity of the sandwich assay to the background subtracted fluorescent intensity of the direct label assay. Background subtracted fluorescent intensities of sandwich format assays were all higher than the corresponding direct label format assays, although the extent to which the sandwich assays outperformed the direct assays was variable for the different cytokines, growth factors and slides examined.

FAST® and ALS Aldehyde slides performed best with sandwich format results, but the narrower standard deviations for FAST® slide measurements indicate greater consistency than the ALS Aldehyde slides. Thus, FAST® slides were deemed most suitable for sandwich assay. ALS Aldehyde slides had the best results from direct label assays performed on all the tested slides. Silylated slides performed moderately on both assays, and SuperEpoxy produced the poorest results overall.

Overall, the sandwich assays outperformed the direct label assays on all four slides examined (FIG. 8). The glass slides had the general array printing problems of spot-to-spot variability and low protein accumulation expected from the printing of capture antibody onto low surface area substrates, while the FAST® slides exhibited greater absorbency of the capture protein and better spot-to-spot consistency. In direct label assays, however, the absorbent FAST® slides also trapped labeled BSA carrier protein in the interstitial space between printed array elements, resulting in a high fluorescent background. Thus, the direct label assays performed comparatively better on the three glass slides tested, particularly Silylated and ALS Aldehyde slides, while sandwich assays performed best on FAST® nitrocellulose membranes.

### Example 16

### Tests Employing FAST® Slides Containing Eight 8×5 Arrays

Antibodies, cytokines, and growth factors. All monoclonal capture antibodies, cytokines, growth factors, and biotinylated detection antibodies were obtained from R&D Systems (Minneapolis, Minn., United States of America).

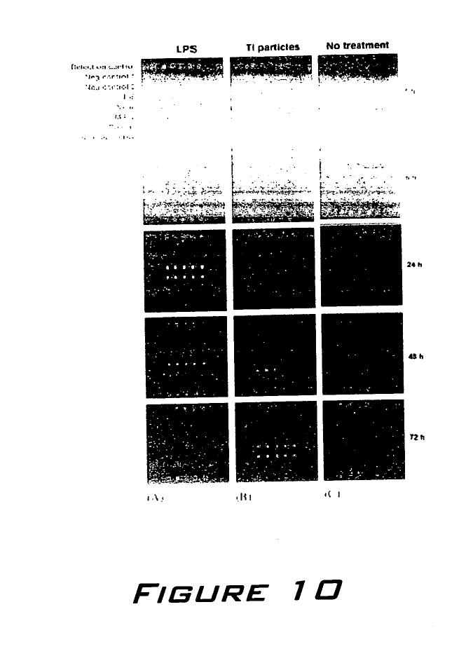

Slides and incubation chambers. The substrates used were 8-pad FAST® slides (glass slides coated with 8 pads of microporous nitrocellulose; Schleicher & Schuell BioScience, Keene, N.H., United States of America). Incubation chambers compatible with the 8-pad FAST® slide were also obtained from Schleicher & Schuell (see FIG. 10). Buffers, including array-printing buffers, blocking buffers and wash buffers were prepared as described herein.

Array fabrication. Each 8-pad FAST® slide was robotically printed with 8 identical 8×5 arrays. For each array, Rows 1 and 8 were biotinylated bovine serum albumin (BSA), regarded as the detection rows. Row 2 was BSA, and Row 3 was a capture antibody for human cytokine, regarded as another negative controls. Rows 4-7 were, respectively, capture antibodies for mouse IL-6, TNF-α, MIP-2, and TGF-β1 (see the top array image of FIG. 10A). A Microsys 5100 microarrayer (Cartesian Technologies, Irvine, Calif., United States of America) was used to print anti-mouse cytokine capture antibodies and control spots on microscope slides. Printing was carried out in an atmospherically controlled chamber with a relative humidity of 70% at room temperature. Chipmaker microarray pins, model CMP4 (TeleChem, Sunnyvale, Calif., United States of America), were used for arraying spots of 160 mm-diameter. Prior to printing, the pin was cleaned in absolute alcohol in an ultrasonic bath for 5 minutes and dried in a stream of nitrogen. Routine optimization and characterization of capture anti-cytokine antibody concentration and non-specific cross reactivity testing was conducted as described herein.

Cell tests. RAW 264.7 mouse monocytes (American Type Culture Collection, Manassas, Va., United States of America) were plated in five 6-well plates at 10⁵ cells/well in 2 ml of Dulbecco's Modified Eagle's Medium (D-MEM; Invitrogen Corp., Carlsbad, Calif., United States of America), supplemented with 10% fetal bovine serum (Sigma-Aldrich Co., St. Louis, Mo., United States of America) and containing 100 U/ml penicillin G and 100 μg/ml streptomycin. Each plate represents a different time point: 1, 6, 24, 48, or 72 hours of culture. After overnight of equilibration and medium renewal, 200 μl of phosphate-buffered saline (PBS; Invitrogen Corp.) solution of sterilized and endotoxin-free Ti particles (8.5 μm in diameter; Sigma-Aldrich Co.) at 50 mg/ml was added into the designated wells. LPS (Sigma-Aldrich Co.) solution was added to the designated wells to a concentration of 10 μg/ml in those wells (positive controls). Untreated cells were negative controls. The cell culture was interrupted at 1, 6, 24, 48, and 72 hours, and cell viability was determined based on exclusion of Trypan Blue staining. The culture media was collected, and then centrifuged at 4° C. and the corresponding supernatants were stored in −70° C. for simultaneous array analysis.

Protein array assay. Silicone incubation chambers were placed over the slides that separated each of the 8 printed arrays. Each pad was incubated with 80 μl blocking buffer for 1 hour. The blocking buffer was removed, and 80 μl of each thawed supernatant was added onto a pad. At the same time, standard solutions containing all the four cytokines were added onto one of the array-containing slides for standard dose-response curves. After a 2 hour incubation, the supernatants were aspirated and washed with wash buffer. Following three aspiration/wash steps, each array was incubated for 1 hour with a cocktail containing the four biotinylated detection antibodies diluted 1:200 from stock concentrations (100 μg/ml). Following a second aspiration/wash cycle, each array was incubated with 80 μl of streptavidin-Cy5 (CALTAG Laboratories, Burlingame, Calif., United States of America) for 30 minutes in the dark. Finally, incubation chambers were discarded and slides were aspirated, washed, and dried in a stream of nitrogen. Dried slides were immediately scanned and imaged using a GENEPIX® 4000B microarray scanner and GENEPIX® 5.0 software (Axon Instruments, Union City, Calif., United States of America). All fluorescence intensities in scanned images were reported as background corrected mean fluorescence intensities of the pixels within the spot ellipse.

### Discussion of Example 16

As disclosed herein, the protein array technique for biomaterial evaluation showed no non-specific crossreactivity. IL-6, TNF-α, and MIP-2 displayed a linear response from 10 to 30,000 pg/ml. TGF-β1 had a linear range of 10-10,000 pg/ml. All calibration curves had correlation coefficient (R²) of at least 0.97.

FIG. 10 shows protein array “snapshots” of the cytokine expression patterns, respectively, for the LPS positive control (FIG. 10A), Ti particles treatment (FIG. 10B), and no treatment negative control (FIG. 10C) at five different time points. Cytokine signals in the LPS group are visually stronger than the corresponding Ti particles group except at 72 hours. There was no significant cytokine signal in the negative control group until 72 hours.

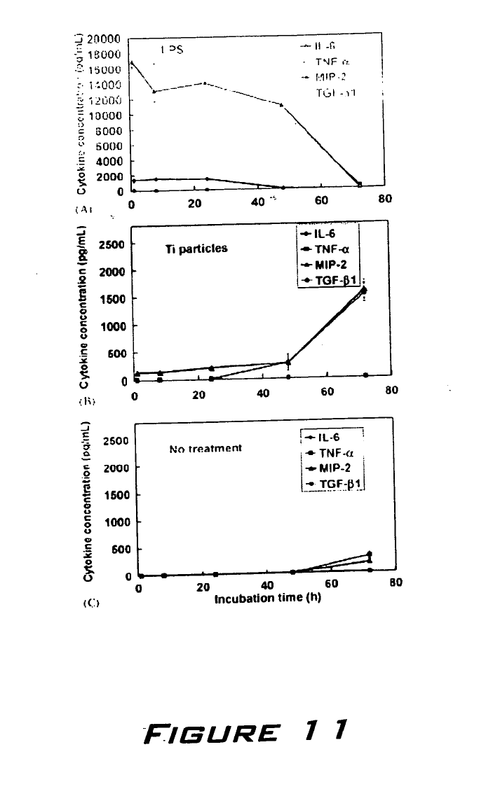

Using calibration standards, FIG. 11 plots the cytokine concentration corresponding to the average array intensity for each experimental condition in FIG. 10. The positive LPS control produced a substantial release of cytokines that decreased with time. The Ti particle treated cells showed a more subtle and gradual increase in the release of cytokines. The negative control showed a weak cytokine release at 72 hours. Among the four cytokines assayed, TNF-α and MIP-2 were the most prominently expressed, while IL-6 showed lower expression levels, and TGF-β1 was undetectable (<10 pg/ml). The absence of TGF-β1 expression might arise because it plays an important role at the wound healing stage rather than in early inflammation. LPS caused an immediate response of mouse monocyte cells, but the cytokine level decreased from very high to undetectable as exposure time increased to 72 hours. Trypan Blue dye exclusion (Table 6) showed 88% cell viability at 1 hour vs. 38% at 72 hours in LPS group. In contrast, the viability of cells in the negative control group remained approximately 90% at all time points. Thus, it appears that cell death resulted in the decrease of cytokine expression with time for the LPS treated cells.

Exposing these monocyte/macrophage cultures to biomaterials, in an attempt to mimic an inflammatory or wound healing response, resulted in cytokine induction. The protocol used reflected the advantages of using protein array. These include, high throughput, multiplex cytokine detection, and sensitivity at a level of pg/ml. The four cytokines employed provided a fingerprint, or profile, for cytokine induction by Ti particles. Other cytokines and growth factors can be employed to reveal more detail of the biomaterial-induced profile. The combination of monocyte/macrophage culture and protein arrays containing expanded anti-cytokine antibody menu can form the basis for sensitive and versatile in vitro biomaterial testing protocols.

TABLE 6
Viability of Murine Monocytes at Different Time Points

| | Viability (%) | | | | | |
|---|---|---|---|---|---|---|
| Group | Start | 1 hr | 6 hr | 24 hr | 48 hr | 72 hr |
| LPS | 92 | 88 | 85 | 62 | 55 | 38 |
| Ti particles | 92 | 91 | 91 | 88 | 88 | 76 |
| No treatment | 92 | 90 | 90 | 92 | 88 | 89 |

### Overview: Examples 17-23

### Biocompatibility Assays

The foregoing cytokine/growth factor microarray methods are used to determine the temporal profile of key inflammatory and reparative macrophage-derived cytokines in cell-material interaction in an in vitro assay. Additionally, cytokine and growth factor expression pattern of the examined biomaterials are compared with the pattern of positive and negative control biomaterials to predict their biocompatibility.

### Example 17

### Array Preparation

Using the methods and techniques set forth in Examples 1-9 (optimized printing buffers, blocking buffers, solid substrates and labels), anti-cytokine arrays are fabricated by robotically spotting different capture antibodies specific for human macrophage-derived cytokines and growth factors onto FAST® slides, which include capture antibodies for human TGF-β1, TGF-β2, TGF-β3, PDGF-BB, IL-1β, IL-1ra, TNF-α, IL-6, VEGF, basic FGF, MCP-1, MIP-1α, IL-4, IL-8, IL-10, EGF, IGF-I, IL-13 and IL-2, and MIP-1β. In sandwich assays, the printed arrays are incubated with the culture medium obtained from monocyte-biomaterials culture, bound by biotin-conjugated detection antibodies, and then detected by streptavidin conjugated Cy3 or Cy5. The operation of the final array is determined by constructing dose-response calibration curves, and testing them in parallel against samples from culture medium.

### Example 18

### Profiling of Cytokines and Growth Factors with a Protein Array Assay

Array-containing slides are blocked with a blocking buffer (PBS containing 3% Tween 20, 5% sucrose and 0.1% NaN3) for 2 hours. Test supernatants frozen in freezer are thawed at same time. After aspiration of the blocking buffer, 80 μl of test supernatant are added onto an array and incubated at room temperature for 2 hours. The aspiration/wash step is repeated 3 times, and 80 μl of detection antibody cocktail (a combination of antibodies directed against human TGF-β1, TGF-β2, TGF-β3, PDGF-BB, IL-1β, IL-1ra, TNF-α, IL-6, VEGF, basic FGF, MCP-1, MIP-1α, IL-4, IL-8, IL-10, EGF, IGF-I, each at 1:250 dilution) are added onto each array and incubated for an additional 1 hour.

After 3 more cycles of aspiration/wash, 80 μl of Streptavidin-Cy5 (SA-Cy5; CalTag Laboratories, Burlingame, Calif., United States of America) at 1:50 dilution are added to each array and incubated for 30 minutes in the dark, washed again as before, and dried in a stream of N₂. Dried slides are immediately scanned and imaged using a GENEPIX® 4000B microarray scanner and GENEPIX® Pro 5.0 software (Axon Instruments, Union City, Calif., United States of America). Data is acquired and analyzed using the same software. For analysis, all the fluorescent intensities are background corrected mean fluorescence intensities of the pixels within the spot ellipse. As for background, median background values are used, because sampling from a uniform distribution for determining the background is assumed. For spots (features), the median values do not accurately reflect spot (feature) intensity because the images are non-uniform. Therefore, mean intensities are used for the spots as described in M. B. Eisen and P. O. Brown, Methods in Enzymology, (1999) 303, 179-205.

Based on the background corrected intensities the concentrations of cytokines and growth factors are deduced from dose-response standard curves. The information corresponding to a test biomaterial is entered into a database as the biosignature for that test biomaterial. Cluster and TreeView (see M. B. Eisen et al., PNAS (1998) 95, 14863-14868) are used to find coordination effects of cytokines and growth factors in the process of cell-biomaterial interactions.

### Example 19

### In vitro Assay of Cell-Biomaterial Interaction

The human THP-1 monocyte cell line (ATCC TIB 202) is obtained from the American Type Culture Collection (Rockville, Md., United States of America). Polymer materials (e.g., bone cements), metals (e.g., dental alloys and orthopedic implants), and ceramics are selected for examination. Polymeric materials that can be used as positive and negative controls for cytotoxicity assays to confirm the performance of the test method are available from the U.S. Pharmacopeial Convention, Inc. (Rockville, Md., United States of America).

The following assay protocol is used:

1. THP-1 monocytes are grown in RPMI 1640 with 10% FBS, 50 μmol/L of β-mercaptoethanol, 100 units/mL penicillin, 100 μg/mL streptomycin, and 2 mmol/L glutamine.

2. The monocytes are plated in 6-well format (10⁶ cells/well) in 1 mL of cell-culture medium.

3. After overnight equilibration, the biomaterials are added to contact directly with the cells. The cells are incubated for another 1, 6, 24, 48, or 72 hours. Lipopolysaccharide (LPS; at 10 μg/ml or 5 μg/ml) is added to positive control wells. The cells are assayed for secretion of cytokines into the cell-culture medium.

4. The growth media are transferred to centrifuge tubes and centrifuged. The supernatant is then assessed for cytokine content or frozen to be assayed subsequently.

5. The adherent cells are washed with PBS, released from the 6-well dishes, and stained for apoptosis using an annexin V-FITC conjugate (R&D Systems; Minneapolis, Minn., United States of America) according to the manufacturer's instructions. Cells are characterized as viable, apoptotic, or necrotic using flow cytometry.

### Example 20

### Temporal Cytokine Profile Determination In Wound Healing Bed of Microdialysis Probes

Cytokine mediators released from macrophages in the wound healing bed of implanted microdialysis probes are collected and profiled temporally using the antibody microarray system disclosed above. The surrounding tissue explanted at strategic times is histologically assessed to correlate acquisition of molecular markers with the composition of the wound healing tissue.

Bare polyethersulfone (PES) microdialysis probes absent polymer coating are implanted in the dorsal subcutis of Sprague Dawley rats. Fractions of microdialysate are collected on days 1, 3, 5, 7, 14, 21, and 28. Histology and SEM analysis of probes explanted at various time points, or upon failure, are used to characterize the level of neovascularization and the composition of the surrounding tissue.

### Example 21

### Probe Implantation

Rats are immobilized and anesthetized with an intraperitoneal injection of 5 mg/kg Pentobarbital Sodium Injection, USP (Abbott Laboratories, North Chicago, Ill., United States of America). Three probes are implanted percutaneously in the dorsal subcutis in each of twenty male Sprague Dawley rats. Probes are placed 5-7 cm beyond the scapular region on either side of the spine in the dorsal subcutus with the probe tips pointed toward the tail and the inflow and outflow exiting through the skin at the base of the neck. The portions of the inflow and outflow exterior to the rat are cut to approximately 2.5 cm. Between measurements, the inflow and outflow are united with a fluid filled connector (CMA/Microdialysis, North Chelmsford, Mass.) so as to avoid contamination and evaporation, and the rat is allowed to move freely.

### Example 22

### In Vivo Sampling

Rats are anesthetized during all microdialysate collections. Probes are perfused with Ringer's solution at 2.0 μl/min with a precision syringe pump (CMA/Microdialysis, North Chelmsford, Mass., United States of America) for all measurements. At designated periods, four 20 minute aliquots are collected from each probe, after a 20 minute equilibration period. At the same time, corresponding blood samples (50 μl) are taken from the tip of the tail. All samples are analyzed with a YSI 23A Glucose Meter (Yellow Springs Instrument Co., Yellow Springs, Ohio, United States of America) or a CMA/600 Microdialysis Analyzer (CMA/Microdialysis). The cytokines are analyzed using the microarray methods disclosed above.

### Example 23

### Explant Procedures

Histological analysis, SEM, and in vitro recalibration are used to assess the wound healing reaction around the probes excised from the tissue on the day of implantation and on designated days subsequent to implantation. For histology, probes are surgically explanted with the adjacent tissue intact. Specimens are fixed in 10% buffered formalin, dehydrated, mounted in paraffin, cut into 6 μm sections, and stained with Hematoxylin & Eosin and Massons Trichrome (n=5 per material type). SEM samples are subsequently dehydrated in ethanol, dried with a critical point dryer, coated with gold and palladium, and viewed with a Philips 501 Scanning Electron Microscope (Eindhoven, Netherlands).

### Example 24

### Customization of a Biomaterial Choice in a Subject

Monocytes are harvested from a subject requiring an implant of a biomaterial prior to implant surgery. These monocytes are exposed to various orthopedic biomaterials that would be appropriate to address the subject's condition. The protein array system described in Example 18 is used to create a biosignature of cytokines and growth factors specific to the subject's responses to different biomaterials. Using this biosignature, the most suitable biomaterial is selected for implantation into the subject, in some cases by using a database that includes the biosignature for that test biomaterial.

It will be understood that various details of the claimed subject matter can be changed without departing from the scope of the claimed subject matter. Furthermore, the foregoing description is for the purpose of illustration only, and not for the purpose of limitation.

1. A method of establishing a profile of one of host organism response to foreign, implanted material, wound healing, and both host organism response to foreign, implanted material and wound healing, comprising:
(a) collecting a biological sample selected from the group consisting of (i) fluid from interstitial space between an implanted biomaterial and host organism tissue, (ii) supernatant from a cell culture to which biomaterial has been exposed, and (iii) a wound;
(b) contacting the biological sample with at least one microarray for the detection of cytokines and growth factors associated with one of host organism response to foreign, implanted material, wound healing, and both host organism response to foreign, implanted material and wound healing, the microarray comprising a plurality of capture antibody samples immobilized on a solid substrate to form a plurality of array elements, wherein:
(i) each capture antibody sample comprises an anti-cytokine or an anti-growth factor capture antibody in a printing buffer solution; and
(ii) each anti-cytokine or an anti-growth factor capture antibody specifically binds a cytokine or growth factor associated with one of host organism response to foreign, implanted material, wound healing, and both host organism response to foreign, implanted material and wound healing;
(c) detecting binding to the microarray of at least one cytokine or growth factor associated with one of host organism response to foreign, implanted material, wound healing, and both host organism response to foreign, implanted material and wound healing, wherein the binding indicates the presence in the biological sample of a cytokine or growth factor associated with one of host organism response to foreign, implanted material, wound healing, and both host organism response to foreign, implanted material and wound healing; and
(d) establishing a profile of one of host organism response to foreign, implanted material, wound healing, and both host organism response to foreign, implanted material and wound healing based on the binding.

2. The method according to claim 1, wherein the collecting step is carried out by microdialysis.

3. The method according to claim 1, wherein biomaterial is material used for an implantable sensor.

4. The method according to claim 1, wherein the wound is the wound-bed interstitial space between a bioimplant and tissue.

5. The method according to claim 1, wherein the contacting step is proceeded by a blocking step, wherein the microarray is contacted with a blocking buffer.

6. The method according to claim 1, wherein the contacting and detecting steps are carried out in at least one immunoassay format.

7. The method according to claim 1, wherein the immunoassay format is selected from the group consisting of direct label immunoassay formats and sandwich immunoassay formats.

8. The method according to claim 7, wherein the immunoassay format is a direct-label immunoassay format.

9. The method according to claim 7, wherein the immunoassay format is a sandwich immunoassay format.

10. The method according to claim 9, wherein the immunoassay format is a sandwich immunoassay format, and the contacting and detecting steps are carried out by:
(a) incubating the biological sample with the microarray, wherein capture antibodies immobilized onto the microarray specifically bind cytokines or growth factors present in the biological sample;
(b) contacting the microarray with a plurality of biotinylated detection antibodies, wherein the detection antibodies specifically bind cytokines or growth factors bound to the capture antibodies;
(c) contacting the microarray with a fluorescent detectable label;
and
(d) imaging the fluorescent detectable label.

11. The method of claim 1, wherein the concentration of the anti-cytokine or an anti-growth factor capture antibody in a printing buffer solution is from about 250 μg/ml to about 500 μg/ml.

12. The method of claim 1, wherein the binding to the microarray of at least one cytokine or growth factor associated with host organism response to foreign, implanted material, healing is correlated with an assessment of biocompatibility.

13. The method of claim 12, wherein the correlation is carried out by:
quantitating the binding of at least one cytokine or growth factor associated with foreign, implanted material; and then comparing the quantitation of binding to a quantity of cytokine or growth factor known to correspond to an indication of biocompatibility.

14. The method of claim 1, comprising comparing the profile with one or more reference profiles.

15. The method of claim 14, comprising selection a foreign, implantable material for implantation into a subject based on the comparing of the profile with one or more reference profiles.

16. A kit for establishing a profile of one of host organism response to foreign, implanted material, wound healing, and both host organism response to foreign, implanted material and wound healing comprising:
(a) at least one microarray for the detection of cytokines and growth factors associated with one of host organism response to foreign, implanted material, wound healing, and both host organism response to foreign, implanted material and wound healing, wherein the microarray comprises a plurality of capture antibody samples immobilized on a solid substrate to form a plurality of array elements, wherein:
(i) each capture antibody sample comprises an anti-cytokine or an anti-growth factor capture antibody in a printing buffer solution; and
(ii) each anti-cytokine or an anti-growth factor capture antibody specifically binds a cytokine or growth factor associated with one of host organism response to foreign, implanted material, wound healing, and both host organism response to foreign, implanted material and wound healing;
(b) at least one reagent useful for the detection of cytokines and growth factors associated with one of host organism response to foreign, implanted material, wound healing, and both host organism response to foreign, implanted material and wound healing; and
(c) instructions for establishing a profile of one of host organism response to foreign, implanted material, wound healing, and both host organism response to foreign, implanted material and wound healing based on the binding.

17. The kit according to claim 16, wherein the plurality of array elements comprises array elements ranging in number from at least five array elements to at least seventeen array elements.

18. The kit according to claim 16, wherein the solid substrate is a modified glass slide.

19. The kit according to claim 16, wherein the printing buffer solution comprises about 70% PBS and about 30% glycerol/EDTA.

20. The kit according to claim 16, the concentration of the anti-cytokine or an anti-growth factor capture antibody in a printing buffer solution is from about 250 μg/ml to about 500 μg/ml.

21. The kit according to claim 16, wherein at least one cytokine or growth factor associated with wound healing is selected from the group consisting of TGF-β1, TGF-β2, TGF-β3, PDGF-BB, IL-1β, TNF-α, IL-6, VEGF, basic FGF, MCP-1, MIP-1α, IL-4, IL-8, IL-10, EGF, IGF-I, MIP-1β, IL-1ra, IL-13, and IL-2.

22. The kit according to claim 16, wherein at least one cytokine or growth factor associated with wound healing is selected from the group consisting of IL1-β, TNF-α, VEGF, MIP-1β and TGF-β1.

23. The kit according to claim 16, wherein the volume of each array element is about 1.0 nL.

24. The kit according to claim 16, wherein the diameter of each array element ranges from about 100 to 200 μm.

25. The kit according to claim 16, wherein the plurality of capture antibody samples is immobilized onto the solid substrate by robotically spotting the capture antibody samples onto the solid substrate in parallel.

26. The kit according to claim 16, wherein the at least one reagent is selected from the group consisting of blocking buffers, detection antibodies, and fluorescent detectable labels.

27. The kit according to claim 16, further comprising written material selected from the group consisting of notification of approved uses of the kit; instructions for carrying out cytokine and/or growth factor detection with the kit; specific information correlating elements of the microarray with particular cytokines or growth factors; specific indicators identifying the significance of the presence or absence of a binding event when a biological sample is exposed to the microarray; and an index or key pursuant to which each element of the array can be correlated to a target cytokine or growth factor.
